(19) United States
(12) Patent Application Publication
(10) Pub. No.: US 2002/0072081 A1
(43) Pub. Date: Jun. 13, 2002

(54) Geranylgeranyl transferase inhibitor screening assay

(76) Inventors: Wai-Si Eng, Maple Glen, PA (US); Robert B. Lobell, Penlllyn, PA (US); William C. Lumma, Helena, MT (US); Anthony M. Smith, Green Lane, PA (US)

Correspondence Address: MERCK AND CO INC, P O BOX 2000, RAHWAY, NJ, 070650907

(21) Appl. No.: 09/947,903

(22) Filed: Sep. 6, 2001

Related U.S. Application Data
(60) Provisional application No. 60/230,270, filed on Sep. 6, 2000.

(51) Int. Cl. C12Q 1/48; C 07D 4 3/14; C 07D 4 3/02

(52) U.S. Cl. 435/15; 544/360; 544/370

(57) ABSTRACT

The present invention is directed toward a GGTase-I competitive binding assay which can be used to determine the relative GGTase-I inhibitory potency of test compounds. The present invention is also directed toward radiolabeled geranylgeranyl-protein transferase type-I inhibitor compounds which are useful to label GGTase-I in assays, whether cell-based, tissue-based or in whole animal.

### RELATED APPLICATION

The present patent application claims the benefit of co-pending provisional application Ser. No. 60/230,270, filed Sep. 6, 2000.

### BACKGROUND OF THE INVENTION

The present invention relates to assays and compounds that are useful for determining potency of binding to and inhibition of geranylgeranyl-protein transferase type I (GGTase-I). In particular, this assay provides rapid screening of inhibitors of GGTase-I in-vitro and in-vivo.

Covalent modification by isoprenoid lipids (prenylation) contributes to membrane interactions and biological activities of a rapidly expanding group of proteins (Maltese, FASEB J. 4:3319 (1990); Glomset et al., Trends Biochem. Sci. 15:139 (1990)). Geranylgeranyl Transferase Type-I (GGTase-I) transfers a geranylgeranyl group from the prenyl donor geranylgeranyl diphosphate to the cysteine residue of substrate proteins containing a C-terminal CAAX-motif in which “A” is any amino acid, including an aliphatic amino acid, and the “X” residue is leucine (Clarke, Ann. Rev. Biochem. 61:355 (1992); Casey, J. Lipid. Res. 330:1731 (1992)). Known targets of GGTase-I include γ-subunits of brain heterotrimeric B proteins and Ras related small GTP-binding proteins.

The Ras protein is part of a signaling pathway that links cell surface growth factor receptors to nuclear signals initiating cellular proliferation. Biological and chemical studies of Ras action indicate that Ras functions like a G-regulatory protein. Activation of Ras leads to activation of multiple intracellular signal transduction pathways, including MAP Kinase and Rho/Rac (Joneson et al., Science 271:810-812).

Mutated ras genes are found in many human cancers, including colorectal carcinoma, exocrine pancreatic carcinoma, and myeloid leukemias. The protein products of these genes are defective in their GTPase activity and constitutively transmit a growth stimulatory signal.

Mammalian cells express four types of Ras proteins (H-, N-, K4A-, and K4B-Ras) among which K4B is the most frequently mutated form of Ras in human cancers.

Farnesylation of Ras is required for Ras oncogenic activity. Thus potent inhibitors of farnesyl transferase (FTase) have been designed as potential anticancer drugs (Lemer et al., J. Biol. Chem. 270:26770-26773 (1995). FTase-inhibitors are weak inhibitors of K4B-Ras. K4B-Ras oncogenic processing and signaling is, however, blocked by the combination of inhibitors of Frase and GGTase-I. Thus, GGTase-I inhibitors are potential anticancer agents in humans.

This has led to a search for specific inhibitors of GGTase-I and selective inhibitors of GGTase-I have been previously disclosed (see for example U.S. Pat. No. 5,470,832, issued Nov. 28, 1995). Other compounds have been described as selective inhibitors of GGTase-I (see for example PCT Publication No. WO 96/21456). Combinations of a selective inhibitor of FTase and a selective inhibitor of GGTase-I have been disclosed as useful in the treatment of cancer (PCT Publication No. 97/34664).

GGTase-I inhibitors represent a new pharmacological approach to the treatment of cancer that is mechanism-based and does not rely on a cytotoxic mechanism of action. Ideally, therapeutically effective doses of GGTase-I inhibitors will not be limited by cytotoxic side effects and these compounds will have a much larger therapeutic window than currently available antitumor drugs.

Previously, determining the relative potency of compounds for GGTase-I inhibition in cells was accomplished by analysis of inhibition of geranylgeranylation of a substrate protein such as Rap 1a by immunoblot analysis. This invention offers the ability to directly measure inhibition of GGTase-I in cells via a competitive binding assay, and could be a higher throughput, faster, more accurate screening assay.

It is, therefore, an object of this invention to develop radiolabeled GGTase-I inhibitor compounds that would be useful in assays, both in vitro and in vivo, for labeling the enzyme and for competing with unlabeled GGTase-I inhibitors. It is a further object of this invention to develop novel assays which comprise such radiolabeled compounds.

It is also the object of this invention to provide for a radiolabeled GGTase-I inhibitor compound which is optimized for in vivo imaging and is therefore useful for determining the appropriate clinical doses of GGTase-I inhibitor which will be used to assess antitumor efficacy in humans.

### SUMMARY OF THE INVENTION

The present invention is directed toward a GGTase-I competitive binding assay which can be used to determine the relative GGTase-I inhibitory potency of test compounds. This competitive binding assay can be cell-based, tissue-based or modified for use in the whole animal. Also disclosed are radiolabeled GGTase-I inhibitor compounds that are useful in this assay for determining the cellular potency of test compounds in inhibiting GGTase-I.

### BRIEF DESCRIPTION OF THE FIGURES

FIG. 1: Non-Specifc Binding of [³H]-Compound A in Hras2b/rat1 Cells

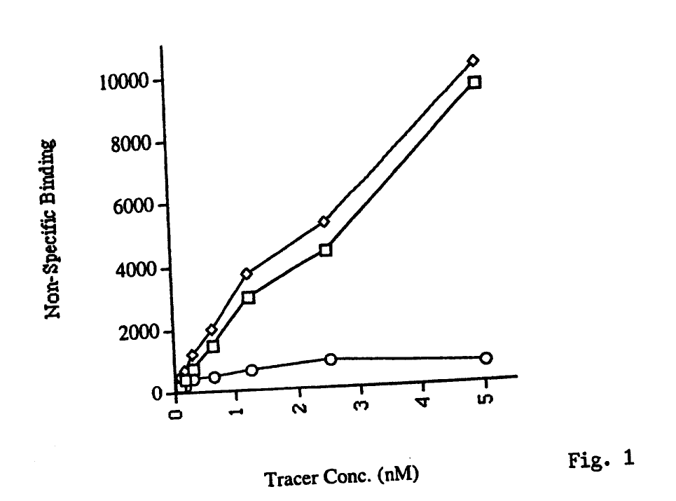

Relative radioemissions from cell lysates of Hras2b/rat1 cells treated with varying concentrations of [³H]-Compound A in the absence (-¤-) and presence (-⋄-) of 1000 fold molar excess of the unlabeled Compound A (procedure described in the Examples). The difference in the two plots (-O-) is the specific binding of [³H]-Compound A as a function of varying concentrations of the radiolabeled GGTase-I inhibitor.

FIG. 2: Specific Binding of [³H]-Compound A in Hras2b/rat1 Cells

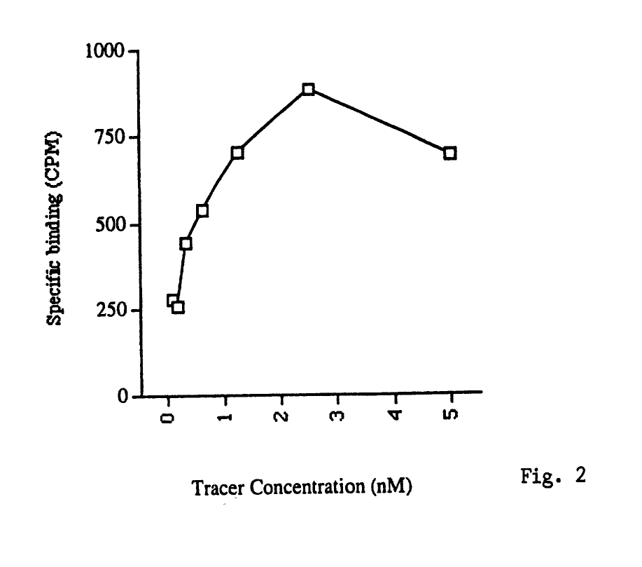

Plot of the specific binding of [³H]-Compound A to geranylgeranyl transferase type I as a function of varying concentrations of the radiolabeled GGTase-I inhibitor.

FIG. 3: Determinations of Relative IC50s of a Selective Inhibitor of GGTase-I and a Dual Inhibitor of GGTase-I and FPTase

Plots of radioemissions from cell lysates of Hras2b/rat1 cells which were incubated with 0.3 nM [³H]-Compound A and various concentrations of the test compounds: unlabeled compound A (O) and 1-(3-Chlorophenyl)-4-[1-(4-cyanobenzyl)-5-imidazolylmethyl]-2-piperazinone (described in U.S. Pat. No. 5,856,326 granted on Jan. 5, 1999) (Δ).

### DETAILED DESCRIPTION OF THE INVENTION

The instant invention is directed to an assay that measures the competitive binding of a GGTase-I inhibitor test compound and a radiolabeled GGTase-I inhibitor for binding to GGTase-I binding sites in living cells. Such an assay for example would comprise the steps of:

a) culturing monolayers of cells;

b) exposing a monolayer of cells to growth media containing the radiolabeled GGTase-I inhibitor in the presence or absence of the test compound;

c) washing the cells;

d) counting the radiation emitted by the cells; and

e) comparing the radiation emitted by cells exposed to the radiolabeled GGTase-I inhibitor and the test compound to the radiation emitted by cells exposed to only the radiolabeled GGTase-I inhibitor.

This invention is a competitive radioligand binding assay for GGTase-I that can be used for the determination of the relative inhibitory activity of compounds for the GGTase-I enzyme in living cells. The assay uses a radiolabeled GGTase-I inhibitor, and measures the amount of binding of this radioligand to GGTase-I in cells. By incubating this radioligand with increasing concentrations of unlabeled test compound, one can determine the amount of specifically bound test compound in a population of cells, which represents a specific high affinity interaction with GGTase-I.

In an embodiment of the instant invention, the assay involves growing a cell line in 24-well cell culture plates as an adherent population of cells until the cell culture is nearly confluent. The concentration of radioligand required to achieve half-maximal specific binding to cells is determined by adding the radioligand at varying concentrations to cell culture media, and incubating this mixture with the cells for a defined period of time. To determine the level of non-specific binding of the radioligand, excess unlabeled GGTase-I inhibitor (unlabeled radioligand) is added to some of the wells. After incubation, the unbound radioligand is removed from the cells. The cell layer is quickly rinsed to wash away unbound radiotracer. The cell layer is then solubilized and analyzed. Having determined the concentration of radioligand that achieves half maximal binding to cells, one can determine the relative IC₅₀s of test compounds by incubating varying concentrations of test compounds with the appropriate concentration of radioligand, and perform the cell incubations, washes, and solubilization steps as described.

Suitable radionuclides that may be incorporated in the instant compounds include 3H (also written as T), ¹¹C, ¹⁸F, ¹²⁵I, ⁸²Br, ¹²³I, ¹³¹I, ⁷⁵Br, ¹⁵O, ¹³N, ²¹¹At or ⁷⁷Br. The radionuclide that is incorporated in the instant radiolabeled compounds will depend on the specific analytical or pharmaceutical application of that radiolabeled compound. Thus, for in vitro GGTase-I labeling and competition assays, inhibitor compounds that incorporate ³H, ¹²⁵I or ⁸²Br will generally be most useful. For diagnostic imaging agents, inhibitor compounds that incorporate a radionuclide selected from ¹¹C, ¹⁸F, ¹²³I, ¹³¹I, ⁷⁵Br, ⁷⁶Br or ⁷⁷Br are preferred. In certain applications incorporation of a chelating radionuclide such as Tc^{99m} may also be useful.

The radiolabeled GGTase-I inhibitor should bind with a high affinity to GGTase-I. Preferably the labeled Inhibitor has an IC₅₀<50 nM. When the farnesyl transferase inhibitory activity of the radiolabeled GGTase-I inhibitor is high (IC₅₀<100 nM), an unlabeled potent FTI may be used to block binding to farnesyl transferase.

Because the GGTase-I that is interacting with the labeled inhibitor is generally cellular GGTase-I, the labeled inhibitor of the instant invention must be diffuseable across the cell membrane and remain diffuseable after binding to GGTase-I to avoid intracellular accumulation of labeled inhibitor which might contribute to greater assay background noise. Therefore, it is preferred that the labeled inhibitor have a lipophilicity (partition coefficient) in the range of about 1.0 to 3.0. It is also preferred that the labeled inhibitor chosen is generally free from nonspecific intracellular interactions that would alter the compounds permeability or effect its GGTase-I binding affinity. Therefore, while many GGTase-I inhibitors have been described that incorporate a thiol moiety, the nonspecific interactions associated with such a moiety disfavor those inhibitors. Similarly, ester prodrugs which exhibit potent intercellular GGTase-I inhibitory activity only upon conversion to their corresponding acid within the cell are also disfavored because the conversion to the active acid would alter the permeability of the labeled inhibitor.

Radiolabeled GGTase-I inhibitor compounds, when labeled with the appropriate radionuclide, are potentially useful for diagnostic imaging, basic research, and radiotherapeutic applications. Specific examples of possible diagnostic imaging applications include:

1. Location of primary and metastatic tumors of the pancreas; exocrine tumors;

2. Diagnosis and staging of colorectal carcinoma.

3. Diagnosis and staging of myeloid leukemia.

4. Diagnosis and staging of neurological tumors.

5. Diagnosis and staging of the benign proliferative disorder associated with NF-1.

6. Diagnosis of neointimal formation resulting from percutaneous transluminal coronary angioplasty.

7. Diagnosis and staging of polycystic kidney disease.

Specific examples of possible radiotherapeutic applications include:

1. Radioimmunoassay of GGTase-I inhibitors.

2. Radioimmunoassay to determine the concentration of GGTase-I in a tissue sample.

3. Autoradiography to determine the distribution of GGTase-I in a mammal or an organ or tissue sample thereof.

For the use of the instant compounds as diagnostic imaging agents the radiolabeled compounds may be administered to mammals, preferably humans, in a pharmaceutical composition, either alone or, preferably, in combination with pharmaceutically acceptable carriers or diluents, optionally with known adjuvants, such as alum, in a pharmaceutical composition, according to standard pharmaceutical practice. Such compositions can be administered orally or parenterally, including the intravenous, intramuscular, intraperitoneal, subcutaneous, rectal and topical routes of administration. Preferably, administration is intravenous.

For intramuscular, intraperitoneal, subcutaneous and intravenous use, sterile solutions of the labeled compound are usually prepared, and the pH of the solutions should be suitably adjusted and buffered. For intravenous use, the total concentration of solutes should be controlled in order to render the preparation isotonic. For oral use of a diagnostic imagining combination according to this invention, the selected combination or compounds may be administered, for example, in the form of tablets or capsules, or as an aqueous solution or suspension. In the case of tablets for oral use, carriers which are commonly used include lactose and corn starch, and lubricating agents, such as magnesium stearate, are commonly added. For oral administration in capsule form, useful diluents include lactose and dried corn starch. When aqueous suspensions are required for oral use, the active ingredients are combined with emulsifying and suspending agents. If desired, certain sweetening and/or flavoring agents may be added.

Suitable compositions of this invention include aqueous solutions comprising compounds of this invention and pharmacologically acceptable carriers, e.g., saline, at a pH level, e.g., 7.4. The solutions may be introduced into a patient's bloodstream by local bolus injection.

As used herein, the term “composition” is intended to encompass a product comprising the specified ingredients in the specific amounts, as well as any product which results, directly or indirectly, from combination of the specific ingredients in the specified amounts.

When a radiolabeled compound according to this invention is administered into a human subject, the amount required for diagnostic imaging will normally be determined by the prescribing physician with the dosage generally varying according to the age, weight, and response of the individual patient, as well as the quantity of emission from the radionuclide. However, in most instances, an effective amount will be the amount of compound sufficient to produce emissions in the range of from about 1-5 mCi.

In one exemplary application, administration occurs in an amount of radiolabeled compound of between about 0.005 μg/kg of body weight to about 50 μg/kg of body weight per day, preferably of between 0.02 μg/kg of body weight to about 3 μg/kg of body weight.

A particular analytical dosage that comprises the instant composition includes from about 0.5 μg to about 100 μg of a labeled GGTase-I inhibitor. Preferably, the dosage comprises from about 1 μg to about 50 μg of a radiolabeled GGTase-I inhibitor.

The following illustrative procedure may be utilized when preforming PET imaging studies on patients in the clinic:

The patient is fasted for at least 12 hours allowing water intake ad libitum, and is premedicated with 0.3-0.4 mL Acepromazine injected i.m. on the day of the experiment. A 20 G two inch venous catheter is inserted into the contralateral ulnar vein for radiotracer administration.

The patient is positioned in the PET camera and a tracer dose of [¹⁵O]H₂O administered via i.v. catheter. The image thus obtained is used to insure that the patient is positioned correctly to include liver, kidneys, tumors and pancreas. Subsequently the [¹¹C] radiolabeled GGTase-I inhibitor (<20 mCi) is administered via i.v. catheter. Following the acquisition of the total radiotracer image, an infusion is begun of the GGTase-I inhibitor which is being clinically evaluated (clinical candidate) at one of three dose rates (0.1, 1 or 10 mpk/day). After infusion for 2.5 hrs, the [¹¹C] radiolabeled GGTase-I inhibitor is again injected via the catheter. Images are again acquired for up to 90 min. Within ten minutes of the injection of radiotracer and at the end of the imaging session, 1 ml blood samples are obtained for determining the plasma concentration of the clinical candidate.

For uninhibited distribution of radiotracer, regions of interest (ROIs) are drawn on the reconstructed image includes the tumor, kidney cortex and a region of liver which is removed from the gallbladder images. These regions are used to generate time activity curves obtained in the absence of inhibitor or in the presence of the clinical candidate at the various infusion doses examined. Data are expressed as radioactivity per unit time per unit volume (μCi/cc/mCi injected dose). Inhibition curves are generated from the data obtained in a region of interest obtained starting at 70 min. post-injection of radiotracer. At this time, clearance of non-specific binding has reached steady state. The ID₅₀ values were obtained by curve fitting the dose-rate/inhibition curves with equation iii:

B=A₀−A₀*I/(ID₅₀+I)+NS (iii)

where B is the %-Dose/g of radiotracer in tissues for each dose of clinical candidate, A₀ is the specifically bound radiotracer in a tissue in the absence of clinical candidate, I is the injected dose of inhibitor, ID₅₀ is the dose of clinical candidate which inhibits 50% of specific radiotracer binding to GGTase-I, and NS is the amount of non-specifically bond radiotracer.

The present invention also is directed toward radiolabeled geranylgeranyl-protein transferase type I (GGTase-I) inhibitor compounds which are useful for labeling GGTase-I in assays, whether cell-based, tissue-based or in whole animal. Compounds with IC₅₀s against FTase less than 200 nM (a strong FTase inhibitor) may be employed to wash out FTase binding/activity, as noted above. Preferably, the present compounds have an IC₅₀ of at least 50 nM or less activity against GGTase-I.

The following radiolabeled compounds, which are inhibitors of GGTase-I, are particularly useful as radiotracers and as components in the assay of this invention:

i) a compound of the formula A:

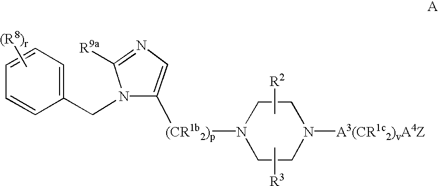

wherein:

R^{1b} is independently selected from:

a) hydrogen,

b) aryl, heterocycle, cycloalkyl, R¹⁰O—, —N(R¹⁰)₂ or C₂-C₆ alkenyl,

c) C₁-C₆ alkyl unsubstituted or substituted by aryl, heterocycle, cycloalkyl, alkenyl, R¹⁰O—, or —N(R¹⁰)₂;

R^{1c} is independently selected from:

a) hydrogen,

b) R¹⁰O—, R¹⁰C(O)NR¹⁰—, (R¹⁰)₂NC(O)—, R¹⁰₂N—C(NR¹⁰)—, CN, NO₂, R¹¹C(O)—, N₃, —N(R¹⁰)₂ or R¹¹OC(O)NR¹⁰—,

c) unsubstituted or substituted C₁-C₆ alkyl wherein the substitutent on the substituted C₁-C₆ alkyl is selected from R¹⁰O—, R¹⁰C(O)NR¹⁰—, (R¹⁰)₂NC(O)—, R¹⁰₂N—C(NR¹⁰)—, CN, R¹⁰C(O)—, N₃, —N(R¹⁰)₂ and R¹¹OC(O)—NR¹⁰—;

R³ is selected from H and CH₃;

R² is selected from H;

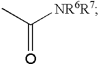

or C₁₋₅ alkyl, unbranched or branched, unsubstituted or substituted with one or more of:

1) aryl,

2) heterocycle,

3) OR⁶,

4) SR^{6a}, SO₂R^{6a}, or

5)

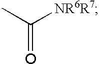

and R² and R³ are optionally attached to the same carbon atom;

R⁶ and R⁷ are independently selected from: H; C₁₋₄ alkyl, C₃₋₆ cycloalkyl, aryl, heterocycle, unsubstituted or substituted with:

a) C₁₋₄ alkoxy,

b) halogen, or

c) aryl or heterocycle;

R^{6a} is selected from: C₁₋₄ alkyl or C₃₋₆ cycloalkyl, unsubstituted or substituted with:

a) C₁₋₄ alkoxy,

b) halogen, or

c) aryl or heterocycle;

R⁸ is independently selected from:

a) hydrogen,

b) C₁-C₆ alkyl, C₂-C₆ alkenyl, C₂-C₆ alkynyl, C₁-C₆ perfluoroalkyl, F, Cl, R¹⁰O—, R¹⁰C(O)NR¹⁰—, CN, NO₂, (R¹⁰)₂N—C(NR¹⁰)—, R¹⁰C(O)—, —N(R¹⁰)₂, or R¹¹OC(O)NR¹⁰—, and

c) C₁-C₆ alkyl substituted by C₁-C₆ perfluoroalkyl, R¹⁰O—, R¹⁰C(O)NR¹⁰—, (R¹⁰)₂N—C(NR¹⁰)—, R¹⁰C(O)—, —N(R¹⁰)₂, or R¹¹C(O)NR¹⁰—;

R^{9a} is hydrogen or methyl;

R¹⁰ is independently selected from hydrogen, C₁-C₆ alkyl, benzyl and aryl;

R¹¹ is independently selected from C₁-C₆ alkyl and aryl;

A³ is selected from: —C(O)—, —C(O)NR¹⁰— or —C(O)O—;

A⁴ is selected from: bond and —O-;

Z is substituted C₅-C₇ cycloalkyl, wherein the substituted C₅-C₇ cycloalkyl is substituted with one or more C₁₋₄ alkyl moieties and is optionally substituted with one or two moieties selected from the following:

a) C₁₋₄ alkoxy,

b) NR⁶R⁷,

c) C₃-6 cycloalkyl,

d) —NR⁶C(O)R⁷,

e) HO,

f) —S(O)ₘR^{6a},

g) halogen, or

h) perfluoroalkyl;

mis 0, 1 or 2;

p is 1, 2 or 3;

r is 0 to 5, and

v is 0, 1, 2 or 3;

and wherein at least one radionuclide or ³H-methyl is present in the molecule;

ii) a compound of the formula B:

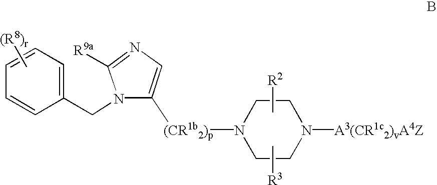

wherein:

R^{1b}, R^{1c}, R², R³, R⁶, R⁷, R^{6a}, R⁸, R^{9a}, R¹⁰, R¹¹, A³, A⁴, m, p, rand v are as defined above for the compound of the formula A; Z is an unsubstituted or substituted C₇-C₁₀ multicyclic alkyl ring, wherein the substituted C₇-C₁₀ multicyclic alkyl ring is substituted with one or two moieties selected from the following:

a) C₁₋₄ alkoxy,

b) NR⁶R⁷,

c) C₃₋₆ cycloalkyl,

d) —NR⁶C(O)R⁷,

e) HO,

f) —S(O)ₘR^{6a},

g) halogen,

h) perfluoroalkyl, and

i) C₁₋₄ alkyl;

C₇-C₁₀ multicyclic alkyl ring is selected from:

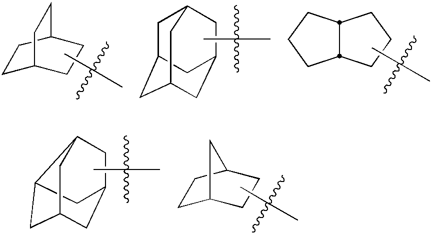

and wherein at least one radionuclide or ³H-methyl is present in the molecule;

iii) a compound of the formula C:

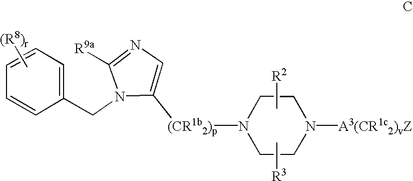

wherein:

R^{1c}, R³, R⁶, R⁷, R^{6a}, R⁸, R¹⁰, R¹¹, m, p and r are as defined above for the compound of the formula A;

R^{1b} is independently selected from:

a) hydrogen, P1 b) aryl, heterocycle, cycloalkyl, CN, R¹⁰O—, R¹⁰NC(O)—, —N(R¹⁰)₂ or C₂-C₆ alkenyl,

c) C₁-C₆ alkyl unsubstituted or substituted by aryl, heterocycle, cycloalkyl, alkenyl, R¹⁰O—, or —N(R¹⁰)₂;

R² is selected from H;

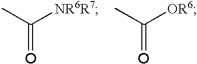

or C₁₋₅ alkyl, unbranched or branched, unsubstituted or substituted with one or more of:

1) aryl,

2) heterocycle,

3) OR⁶,

4) SR^{6a}, SO₂R^{6a}, or

5)

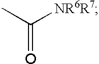

and R² and R³ are optionally attached to the same carbon atom;

R^{9a} is hydrogen, C₁-C₆ alkyl or chloro;

A³ is selected from: —C(O)—, —C(O)NR¹⁰—, —C(O)O— and S(O)ₘ;

Z is unsubstituted or substituted phenyl, unsubstituted or substituted napthyl, unsubstituted or substituted pyridyl, unsubstituted or substituted quinoline or unsubstituted or substituted 1,2 methylenedioxybenzene; and

v is 0, 1, 2 or 3; provided that v is not 0 if A³ is —C(O)— or S(O)ₘ;

and wherein at least one radionuclide or ³H-methyl is present in the molecule;

iv) a compound of the formula D:

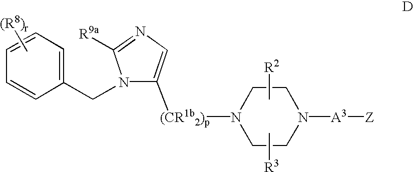

wherein:

R^{1b}, R², R³, R⁶, R⁷ R^{6a}, R⁸, R^{9a}, R¹⁰, R¹¹, A³, m, p, r and v are as defined above for the compound of the formula A;

Z is unsubstituted or substituted C₅-C₁₀ alkyl, wherein the substituted C₅-C₁₀ alkyl is substituted with one or two moieties selected from the following:

a) C₁₋₄ alkoxy,

b) NR⁶R⁷,

c) C₃₋₆ cycloalkyl,

d) —NR⁶C(O)R⁷,

e) —OR¹⁰,

f) —S(O)ₘR^{6a},

g) halogen, or

h) perfluoroalkyl;

and wherein at least one radionuclide or ³H-methyl is present in the molecule;

v) a compound of the formula E:

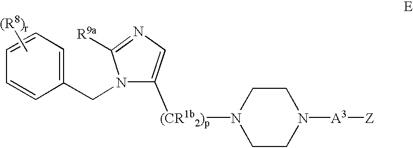

wherein:

R^{1b}, R⁸, R¹¹, p and r are as defined above for the compound of the formula A;

R^{9a} is hydrogen, C₁-C₆ alkyl or chloro;

R¹⁰ is independently selected from hydrogen, C₁-C₆ alkyl, benzyl and aryl;

A³ is —C(O)—;

Z is unsubstituted or substituted phenyl, unsubstituted or substituted napthyl, unsubstituted or substituted pyridyl, unsubstituted or substituted 2,3-dihydrobenzofuran, unsubstituted or substituted quinoline or unsubstituted or substituted isoquinoline;

and wherein at least one radionuclide or ³H-methyl is present in the molecule;

or a pharmaceutically acceptable salt thereof.

Examples of inhibitors of geranylgeranyl protein transferase type I which may be modified to incorporate a radionuclide and be thereby useful in the assay of this invention are as follows:

1-[1-(4′-Cyanobenzyl) imidazol-5-ylmethyl]piperazine-4-carboxylic acid-(3,3,5,5-tetramethyl)cyclohexyl ester

1-[1-(4′-Cyanobenzyl) imidazol-5-ylmethyl]piperazine-4-carboxylic acid-(3,3-dimethyl)cyclohexyl ester

1-[1-(4′-Cyanobenzyl) imidazol-5-ylmethyl]piperazin-3-one-{4-[2-(1,3,3-trimethylcyclohexane)-1-ethyl]}

1-(1-(4-cyanobenzyl)imidazol-5-ylmethyl)-4-(3,3-dimethylcyclohexyloxycarbonyl)-(cis)-2,6-dimethylpiperazine

1-(4-cyanobenzyl)-5-[1-(3,3-dimethylcylohexylacetyl)piperazin-4-ylmethyl]imidazole

1-[1-(4′-Cyanobenzyl) imidazol-5-ylmethyl]piperazine-4-(2-hydroxy-2-cyclohexylacetamide

1-[1-(4′-Cyanobenzyl) imidazol-5-ylmethyl]piperazine-4-carboxylic acid-[(1R,2S,5R)-2-isopropyl-5-methyl]cyclohexyl ester

1-[1-(4′-Cyanobenzyl) imidazol-5-ylmethyl]piperazine-4-carboxylic acid-(2,6-dimethyl)cyclohexylmethyl ester

1-[1-(4′-Cyanobenzyl) imidazol-5-ylmethyl]piperazine-4-carboxylic acid-(2-tertbutyl)cyclohexyl ester

1-[1-(4′-Cyanobenzyl) imidazol-5-ylmethyl]piperazine-4-carboxylic acid-(3-methyl )cyclohexyl ester

1-[1-(4′-Cyanobenzyl) imidazol-5-ylmethyl]4-(2,2-dicyclohexyl)acetyl piperazine

1-[1-(4′-Cyanobenzyl) imidazol-5-ylmethyl]piperazine-4-(2,2-dimethyl-3-isobutenylcyclopropane)carboxamide

1-[1-(4-cyanophenyl)methylimidazol-5-ylmethyl]piperazine-4-cyclohexyloxycarbonyl

R/S 1-(4-Cyanobenzyl)-5-[1-(2-hydroxy-2-(adamant-1-yl)ethyl)-2-oxo-piperazin-4-yl-methyl]imidazole

1-(4-Cyanobenzyl)-5-[1-(2-acetyloxy-2-(adamant-1-yl)ethyl)-2-oxo-piperazin-4-yl-methyl]imidazole

1-[1-(4′-Cyanobenzyl) imidazol-5-ylmethyl]piperazine-4-(N-1-adamantyl)carboxamide

1-[1-(4′-Cyanobenzyl) imidazol-5-ylmethyl]-4-(N-1-adamantyl)carbonyl piperazine

1-(4-Cyanobenzyl)-5-[1-(2-(adamant-1-yl)ethyl)-2-oxo-piperazin-4-yl-methyl]imidazole

1-[1-(4′-Cyanobenzyl) imidazol-5-ylmethyl]-piperazine-4-carboxylic acid (2-norbornane)methyl ester

1-[1-(4′-Cyanobenzyl) imidazol-5-ylmethyl]-4-(2-bicyclo-[2.2.2]-octylcarbonyl)piperazine

1-[1-(4′-Cyanobenzyl) imidazol-5-ylmethyl]-4-cis/trans-(2,6,6-trimethylbicyclo [3.1.1]heptanecarbonyl)-piperazine

1-[1-(4′-Cyanobenzyl) imidazol-5-ylmethyl]-4-[1-phenyl-1-cyclopentylcarbonyl]piperazine

1-[1-(4′-Cyanobenzyl) imidazol-5-ylmethyl]-4-[cyclohexylphenylacetyl]piperazine

1-[1-(4′-Cyanobenzyl) imidazol-5-ylmethyl]-4-[1-(3-methoxyphenyl)-1-cyclopentylcarbonyl]piperazine

1-[1-(4′-Cyanobenzyl) imidazol-5-ylmethyl]-4-[-(3-phenoxyphenyl)-1-cyclopentylcarbonyl]piperazine

1-[1-(4′-Cyano-3-fluorobenzyl) imidazol-5-ylmethyl]-4-[1-(3-hydroxyphenyl)-1-cyclohexylcarbonyl]piperazine

1-[1-(4′-Cyanobenzyl) imidazol-5-ylmethyl] piperazine-4-carboxylic acid-(2,6-dimethoxy)benzyl ester

1-[1-(4′-Cyanobenzyl) imidazol-5-ylmethyl] piperazine-4-(DL-2-hydroxy-2-(o-methoxyphenyl)) acetamide

1-[1-(4′-methylbenzyl) imidazol-5-ylmethyl]-4-[1-(2,6-dimethylbenzyloxycarbonyl]piperazine

1-[1-(4′-Cyanobenzyl) imidazol-5-ylmethyl] piperazine-4-carboxylic acid-(4-nitro)phenyl ester

1-[1-(4′-Cyanobenzyl) imidazol-5-ylmethyl] piperazine-4-(N-3-isopropenyl-1,1-dimethylbenzyl)carboxamide

1-[1-(4′-Cyanobenzyl) imidazol-5-ylmethyl] piperazine-4-(N-2-chlorobenzyl)carboxamide

1-[(4-cyanophenyl)methylimidazol-5-ylmethyl] piperazine-4-2,4-dimethylbenzyloxycarbonyl

1-[1-(4-cyanophenyl)methylimidazol-5-ylmethyl] piperazine-4-(2-methylbenzyloxycarbonyl)

1-[1-(4-cyanophenyl)methylimidazol-5-ylmethyl] piperazine-4-[(3′-methylbenzyloxycarbonyl)

1-[1-(4-cyanophenyl)methylimidazol-5-ylmethyl] piperazine-4-(2′-methoxybenzyloxycarbonyl)

1-[1-(4-cyanophenyl)methylimidazol-5-ylmethyl] piperazine-4-(4′-pyridinemethyloxycarbonyl)

1-[1-(4-cyanophenyl)methylimidazol-5-ylmethyl] piperazine-4-(2′,5′-dimethylbenzyloxycarbonyl)

1-[1-(4′-Cyanobenzyl) imidazol-5-ylmethyl]piperazine-4-carboxylic acid-(2,2-dimethyl)propyl ester

1-(1-(4-cyanobenzyl)imidazol-5-ylmethyl)-4-(N-(1,1,3,3-tetramethyl)-butyl) carboxamide]piperazine

1-[1-(4′-Cyanobenzyl) imidazol-5-ylmethyl]-piperazine-4-carboxylic acid (2,2,5,5-tetramethyl)hexyl ester

1-[1-(4′-Cyanobenzyl) imidazol-5-ylmethyl]-piperazine-4-carboxylic acid (2,2-dimethyl)pent-3-yl ester

1-[1-(4′-Cyanobenzyl) imidazol-5-ylmethyl]piperazine-4-carboxylic acid-(3,3-dimethyl)butyric ester

1-[1-(4′-Cyanobenzyl) imidazol-5-ylmethyl]piperazine-4-(2-hydroxy-4,4-dimethyl)valeramide

1-[1-(4′-Cyanobenzyl) imidazol-5-ylmethyl]piperazine-4-carboxylic acid-(2,2-dimethyl)propyl ester

1-[1-(4′-Cyanobenzyl) imidazol-5-ylmethyl]-4-(2-ethylbutanecarbonyl) piperazine

1-[1-(4′-Cyanobenzyl) imidazol-5-ylmethyl]-piperazine-4-carboxylic acid-(2-t-butoxy)ethyl ester

1-(1-(4-Cyanobenzyl)imidazol-5-ylmethyl]-4-(N-(1,1,3,3-tetramethyl)-butyl) carboxamide]piperazine

1-[1-(4′-Cyanobenzyl) imidazol-5-ylmethyl]-piperazine-4-carboxylic acid (2,2,5,5-tetramethyl)hexyl ester

1-[1-(4′-Cyanobenzyl) imidazol-5-ylmethyl]-piperazine-4-carboxylic acid (2,2-dimethyl)pent-3-yl ester

4-[1-(4-Cyanobenzyl)imidazol-5-ylmethyl]-1-(2-methoxyquinolin-4-oyl)piperazine

4-[1-(4-Cyanobenzyl)imidazol-5-ylmethyl]-1-(2-diethylamino-3-ethoxypyrid-5-oyl)piperazine

4-[1-(4-Cyanobenzyl)imidazol-5-ylmethyl]-1-(3-ethylamino-4-isoquinolinoyl)piperazine

4-[1-(4-Cyanobenzyl)imidazol-5-ylmethyl]-1-(5-bromo-1-naphthoyl)piperazine

4-[1-(4-Cyaniobenzyl)imidazol-5-ylmethyl]-1-[5-(pent-1-ynyl)-1-naphthoyl]piperazine

4-[1-(4-Cyanobenzyl)imidazol-5-ylmethyl]-1-[5-(prop-1-ynyl)-1-naphthoyl]piperazine

4-[1-(4-Cyanobenzyl)imidazol-5-ylmethyl]-1-(5-propyl-1-naphthoyl)piperazine

4-[1-(4-Cyanobenzyl)imidazol-5-ylmethyl]-1-(4-bromo-3-methylbenzoyl)piperazine

4-[1-(4-Cyanobenzyl)imidazol-5-ylmethyl]-1-[3-methyl-4-(prop-1-ynyl)benzoyl]piperazine

4-[1-(4-Cyanobenzyl)imidazol-5-ylmethyl]-1-(3-methyl-4-pentylbenzoyl)piperazine

4-[1-(4-Cyanobenzyl)imidazol-5-ylmethyl]-1-(2-cyclopropyleth-ynyl-5-methoxybenzoyl)piperazine

4-[1-(4-Cyanobenzyl)imidazol-5-ylmethyl]-1-(5-methoxy-2-pent-1-ynylbenzoyl)piperazine

4-[1-(4-Cyanobenzyl)imidazol-5-ylmethyl]-1-(5-chloro-2-cyclohexylethynylbenzoyl)piperazine

4-[1-(4-Cyanobenzyl)imidazol-5-ylmethyl]-1-(5-chloro-2-cyclohexylethylbenzoyl)piperazine

4-[1-(4-Cyanobenzyl)imidazol-5-ylmethyl]-1-(4-indoloyl)piperazine

4-[1-(4-Cyanobenzyl)imidazol-5-ylmethyl]-1-(3,5-dimethylbenzoyl)piperazine

4-[1-(4-Cyanobenzyl)imidazol-5-ylmethyl]-1-(8-quinolinoyl)piperazine

4-[1-(4-Cyanobenzyl)imidazol-5-ylmethyl]-1-(2-ethoxy-1-naphthoyl)piperazine

4-[1-(4-Cyanobenzyl)imidazol-5-ylmethyl]-1-(2-quinolinoyl)piperazine

4-[1-(4-Cyanobenzyl)imidazol-5-ylmethyl]-1-(3-methoxy-4-methylbenzoyl)piperazine

4-[1-(4-Cyanobenzyl)imidazol-5-ylmethyl]-1-(6-diethylamino-pyrid-2-oyl)piperazine

4-[1-(4-Cyanobenzyl)imidazol-5-ylmethyl]-1-(1-isoquinolinoyl)piperazine

or a pharmaceutically acceptable salt or optical isomer thereof.

Specific compounds which may be modified to incorporate a radionuclide and be thereby useful in the assay of this invention are as follows:

(R,S) 1-[1-(4′-Cyanobenzyl) imidazol-5-ylmethyl]piperazine-4-carboxylic acid-(2 R,6R-dimethyl)cyclohexylmethyl ester

[chemical structure]

(R,S) 1-[1-(4′-Cyanobenzyl) imidazol-5-ylmethyl]piperazine-4-carboxylic acid-(2R,6S-dimethyl)cyclohexylmethyl ester

[chemical structure]

1-[1-(4′-Cyanobenzyl) imidazol-5-ylmethyl]piperazine-4-carboxylic acid-(3,3,5,5-tetramethyl)cyclohexyl ester

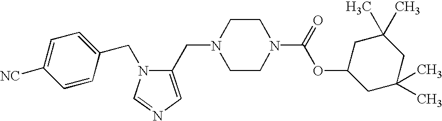

1-(4′-Cyanobenzyl) iniidazol-5-ylmethyl piperazine-4-(2,2-dimethyl-3-isobutenylcyclopropane)carboxamide

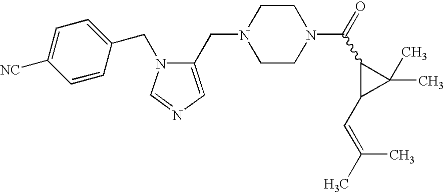

(±) 1-[1-(4′-Cyanobenzyl) imidazol-5-ylmethyl]piperazine-4-carboxylic acid-(3,3-dimethyl)cyclohexyl ester

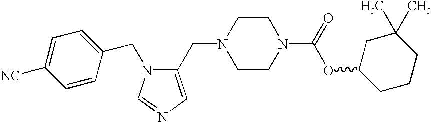

1-[1-(4′-Cyanobenzyl) imidazol-5-ylmethyl] piperazine-4-carboxylic acid-(2,6-dimethoxy)benzyl ester

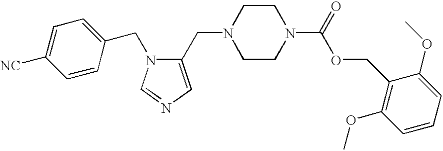

1-[1-(4′-Cyanobenzyl) imidazol-5-ylmethyl]-4-[1-(2,6-dimethylbenzyloxycarbonyl] piperazine

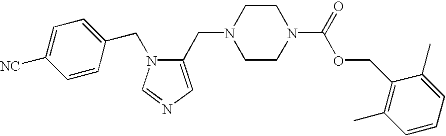

1-[1-(4′-methylbenzyl) imidazol-5-ylmethyl]-4-[1-(2,6-dimethylbenzyloxycarbonyl] piperazine

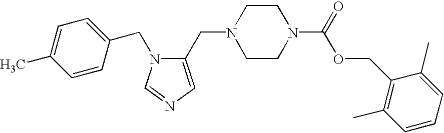

1-[1-(4′-cyanobenzyl) imidazol-5-ylmethyl]-4-[1-(2-ethoxybenzyloxycarbonyl] piperazine

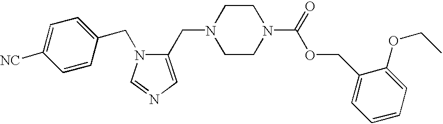

1-[1-(4′-Cyanobenzyl)imidazol-5-ylmethyl]-piperazine-4-(N-2-(ethoxybenzyl)carbamide)

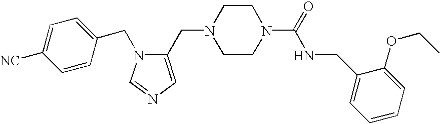

1-[1-1-(4′-Cyanobenzyl) imidazol-5-ylmethyl]-4-{1-[(2-ethoxypyridin-3-yl)methyloxycarbonyl] piperazine

[chemical structure]

4-[1-(4-Cyanobenzyl)imidazol-5-ylmethyl]-1-[3-methyl-4-(prop-1-ynyl)benzoyl]piperazine

[chemical structure]

4-[1-(4-Cyanobenzyl)imidazol-5-ylmethyl]-1-(6-diethylamino-pyrid-2-oyl)piperazine

[chemical structure]

4-[1-(4-Cyanobenzyl)imidazol-5-ylmethyl]-1-(1-naphthoyl)piperazine

[chemical structure]

or the pharmaceutically acceptable salts or optical isomers thereof.

The following radiolabeled compound may be useful in the instant assay:

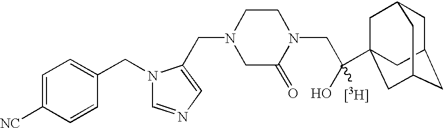

or a pharmaceutically acceptable salt or optical isomer thereof.

Compounds that are selective inhibitors of GGTase-I or are dual inhibitors of GGTase-I and FTase and may therefore be radiolabeled and utilized in the instant invention are described in the following pending applications:

U.S. Ser. No. 09/516756 (filed Mar. 1, 2000) and PCT Appl. No. PCT/US00/05215 (filed Mar. 1, 2000);

U.S. Ser. No. 09/516750 (filed Mar. 1, 2000) and PCT Appl. No. PCT/US00/05216 (filed Mar. 1, 2000);

U.S. Ser. No. 09/516944 (filed Mar. 1, 2000) and PCT Appl. No. PCT/US00/05354 (filed Mar. 1, 2000);

U.S. Ser. No. 09/516757 (filed Mar. 1, 2000) and PCT Appl. No. PCT/US00/05357 (filed Mar. 1, 2000); and

U.S. Ser. No. 09/516945 (filed Mar. 1, 2000) and PCT Appl. No. PCT/US00/05396 (filed Mar. 1, 2000).

Selective inhibitors of GGTase-I have also been described in the following patent applications and publications:

PCT Publication No. WO 97/17070 (May 15, 1997);

U.S. Pat. No. 5,965,539;

Lerner, et al. J. Biol. Chem. 270:26770-26773 (1995);

McGuire et al. J. Biol. Chem. 271:27402-27407 (1996);

Miquel, et al. Cancer Research 57:1846-1850 (1997).

All patents, publications and pending patent applications identified are hereby incorporated by reference.

The compounds of the present invention may have asymmetric centers and occur as racemates, racemic mixtures, and as individual diastereomers, with all possible isomers, including optical isomers, being included in the present invention. When any variable (e.g. aryl, heterocycle, R¹, R² etc.) occurs more than one time in any constituent, its definition on each occurrence is independent at every other occurrence. Also, combinations of substituents/or variables are permissible only if such combinations result in stable compounds.

The term “³H-methyl” represents the moiety —C(³H)₃.

As used herein, “alkyl” is intended to include both branched and straight-chain saturated aliphatic hydrocarbon groups having the specified number of carbon atoms; “alkoxy” represents an alkyl group of indicated number of carbon atoms attached through an oxygen bridge. “Halogen” or “halo” as used herein means fluoro, chloro, bromo and iodo.

As used herein, “cycloalkyl” is intended to include monocyclic saturated aliphatic hydrocarbon groups having the specified number of carbon atoms. Examples of such cycloalkyl groups includes, but are not limited to, cyclopropyl, cyclobutyl, cyclohexyl, cycloheptyl and cyclooctyl.

As used herein, “aryl” is intended to mean any stable monocyclic or bicyclic carbon ring of up to 7 members in each ring, wherein at least one ring is aromatic. Examples of such aryl elements include phenyl, naphthyl, tetrahydronaphthyl, indanyl, biphenyl, phenanthryl, anthryl or acenaphthyl.

The term heterocycle or heterocyclic, as used herein, represents a stable 5- to 7-membered monocyclic or stable 8- to 11-membered bicyclic heterocyclic ring which is either saturated or unsaturated, and which consists of carbon atoms and from one to four heteroatoms selected from the group consisting of N, O, and S, and including any bicyclic group in which any of the above-defined heterocyclic rings is fused to a benzene ring. The term heterocycle or heterocyclic, as used herein, includes heteroaryl moieties. The heterocyclic ring may be attached at any heteroatom or carbon atom which results in the creation of a stable structure. Examples of such heterocyclic elements include, but are not limited to, azepinyl, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, oxazolyl, 2-oxopiperazinyl, 2-oxopiperdinyl, 2-oxopyrrolidinyl, piperidyl, piperazinyl, pyridyl, pyrazinyl, pyrazolidinyl, pyrazolyl, pyridazinyl, pyrimidinyl, pyrrolidinyl, pyrrolyl, quinazolinyl, quinolinyl, quinoxalinyl, tetrahydrofuryl, tetrahydroisoquinolinyl, tetrahydroquinolinyl, thiamorpholinyl, thiamorpholinyl sulfoxide, thiazolyl, thiazolinyl, thienofuryl, thienothienyl, and thienyl.

As used herein, “heteroaryl” is intended to mean any stable monocyclic or bicyclic carbon ring of up to 7 members in each ring, wherein at least one ring is aromatic and wherein from one to four carbon atoms are replaced by heteroatoms selected from the group consisting of N, O, and S. Examples of such heterocyclic elements include, but are not limited to, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, furyl, imidazolyl, indolinyl, indolyl, isochromanyl, isoindolinyl, isoquinolinyl, isothiazolyl, naphthyridinyl, oxadiazolyl, pyridyl, pyrazinyl, pyrazolyl, pyridazinyl, pyrimidinyl, pyrrolyl, quinazolinyl, quinolinyl, quinoxalinyl, tetrahydroisoquinolinyl, tetrahydroquinolinyl, thiazolyl, thienofuryl, thienothienyl, and thienyl.

As used herein in the definition of R² and R³, the term “the substituted group” is intended to mean a substituted C₁₋₈ alkyl, substituted C₂₋₈ alkenyl, substituted C₂₋₈ alkynyl, substituted aryl or substituted heterocycle from which the substituent(s) R² and R³ are selected.

As used herein in the definition of R⁶, R^{6a}, R⁷ and R^{7a}, the substituted C₁₋₈ alkyl, substituted C₃₋₆ cycloalkyl, substituted aroyl, substituted aryl, substituted heteroaroyl, substituted arylsulfonyl, substituted heteroarylsulfonyl and substituted heterocycle include moieties containing from 1 to 3 substituents in addition to the point of attachment to the rest of the compound. Preferably, such substituents are selected from the group which includes but is not limited to F, Cl, Br, CF₃, NH₂, N(C₁-C₆ alkyl)₂, NO₂, CN, (C₁-C₆ alkyl)O—, —OH, (C₁-C₆ alkyl)S(O)ₘ—, (C₁-C₆ alkyl)C(O)NH—, H₂N—C(NH)-, (C₁-C₆ alkyl)C(O)-, (C₁-C₆ alkyl)OC(O)—, N₃, (C₁-C₆ alkyl)OC(O)NH—, phenyl, pyridyl, imidazolyl, oxazolyl, isoxazolyl, thiazolyl, thienyl, furyl, isothiazolyl and C₁-C₂₀ alkyl.

When R² and R³ are combined to form —(CH₂)ᵤ—, and when two R^{1c}s on the same carbon are combined with that carbon to form a C₄-C₆ cycloalkyl, cyclic moieties are formed. Examples of such cyclic moieties include, but are not limited to:

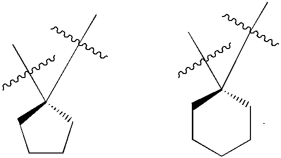

In addition, with respect to R² and R³, such cyclic moieties may optionally include a heteroatom(s). Examples of such heteroatom-containing cyclic moieties include, but are not limited to:

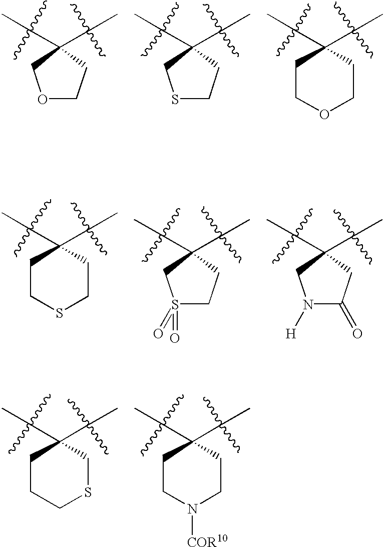

The moiety formed when, in the definition of R⁶, R⁷ and R^{7a}, R⁶ and R⁷ or R⁷ and R^{7a} are joined to form a ring, is illustrated by, but not limited to, the following:

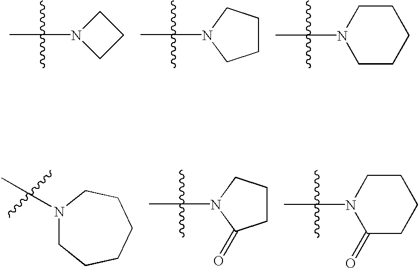

Lines drawn into the ring systems from substituents (such as from R², R³, R⁴ etc.) indicate that the indicated bond may be attached to any of the substitutable ring carbon atoms.

Preferably, R^{1a} and R^{1b} are independently selected from: hydrogen, —N(R¹⁰)₂, R¹⁰C(O)NR¹⁰— or unsubstituted or substituted C₁-C₆ alkyl wherein the substituent on the substituted C₁-C₆ alkyl is selected from unsubstituted or substituted phenyl, —N(R¹⁰)₂, R¹⁰— and R¹⁰C(O)NR¹⁰—.

Preferably, R² is selected from: hydrogen,

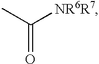

and an unsubstituted or substituted group, the group selected from C₁₋₈ alkyl, C₂₋₈ alkenyl and C₂₋₈ alkynyl; wherein the substituted group is substituted with one or more of:

1) aryl or heterocycle,

2) OR⁶, and

3) SR^{6a}, SO₂R^{6a}.

Preferably, R³ is selected from hydrogen and methyl. Preferably, R⁴ is hydrogen.

Preferably, R⁶ and R⁷ are selected from: hydrogen, unsubstituted or substituted C₁-C₆ alkyl, unsubstituted or substituted aryl and unsubstituted or substituted C₃-C₆ cycloalkyl.

Preferably, R^{6a} is unsubstituted or substituted C₁-C₆.

Preferably, R⁹ is hydrogen or methyl.

Preferably, R¹⁰ is selected from H, C₁-C₆ alkyl and benzyl.

Preferably, A¹ and A² are independently selected from: a bond, —C(O)NR¹⁰—, —NR¹⁰C(O)—, o, —N(R¹⁰)—, —S(O)₂N(R¹⁰)— and —N(R¹⁰)S(O)₂—. Most preferably, A¹ and A² are a bond.

Preferably, A³ is selected from: —C(O)—, —C(O)NR¹⁰— and —C(O)O—.

Preferably, G is H₂.

Preferably, V is selected from heteroaryl and aryl. More preferably, V is phenyl.

Preferably, W is selected from imidazolyl, pyridinyl, thiazolyl, indolyl, quinolinyl, or isoquinolinyl. More preferably, W is imidazolyl and pyridyl.

Preferably, n and r are independently 0, 1, or 2.

Preferably p is 1, 2 or 3.

Preferably s is 0.

Preferably t is 1.

Preferably v is 0, 1 or 2.

Preferably, the moiety

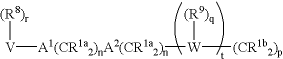

is selected from:

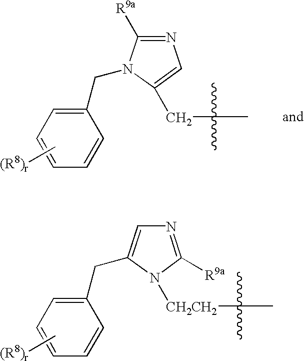

Preferably, the moiety A¹(CR^{1a}₂)ₙA²(CR^{1a}₂)ₙ is not a bond.

It is intended that the definition of any substituent or variable (e.g., R^{1a}, R⁹, n, etc.) at a particular location in a molecule be independent of its definitions elsewhere in that molecule. Thus, —N(R¹⁰)₂ represents —NHH, —NHCH₃, —NHC₂H₅, etc. It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials.

The pharmaceutically acceptable salts of the compounds of this invention include the conventional non-toxic salts of the compounds of this invention as formed, e.g., from non-toxic inorganic or organic acids. For example, such conventional non-toxic salts include those derived from inorganic acids such as hydrochloric, hydrobromic, sulfuric, sulfamic, phosphoric, nitric and the like: and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like.

The pharmaceutically acceptable salts of the compounds of this invention can be synthesized from the compounds of this invention which contain a basic moiety by conventional chemical methods. Generally, the salts are prepared either by ion exchange chromatography or by reacting the free base with stoichiometric amounts or with an excess of the desired salt-forming inorganic or organic acid in a suitable solvent or various combinations of solvents.

Reaction Scemes for Generating Compounds

Synopsis of Schemes 1-20:

The requisite intermediates are in some cases commercially available, or can be prepared according to literature procedures, for the most part. In Scheme 1, for example, the synthesis of 2-cycloalkylalkanoyl substituted piperazines is outlined. Boc-protected amino acids I, available commercially or by procedures known to those skilled in the art, can be coupled to N-benzyl amino acid esters using a variety of dehydrating agents such as DCC (dicyclohexycarbodiimide) or EDC·HCl (1-ethyl-3-(3-dimethylaminopropyl)carbodiimide hydrochloride) in a solvent such as methylene chloride, chloroform, dichloroethane, or dimethylformamide. The product II is then deprotected with acid, for example hydrogen chloride in chloroform or ethyl acetate, or trifluoroacetic acid in methylene chloride, and cyclized under weakly basic conditions to give the diketopiperazine III. Reduction of III with lithium aluminum hydride in refluxing ether gives the piperazine IV, which is protected as the Boc derivative V. The N-benzyl group can be cleaved under standard conditions of hydrogenation, e.g., 10% palladium on carbon at 60 psi hydrogen on a Parr apparatus for 24-48 h. The product VI can be reacted with a suitably substituted carboxylic acid to provide the piperazine VII; a final acid deprotection as previously described gives the intermediate VIII (Scheme 2).

In Scheme 3, for example, boc-protected piperazine VI, available commercially or by procedures known to those skilled in the art, can be coupled to suitable substituted carboxylic acids using a variety of dehydrating agents such as DCC (dicyclohexycarbodiimide) or EDC·HCl (1-ethyl-3-(3-dimethylaminopropyl)carbodiimide hydrochloride) in a solvent such as methylene chloride, chloroform, dichloroethane, or dimethylformamide. The product VII is then deprotected with acid, for example hydrogen chloride in chloroform or ethyl acetate, or trifluoroacetic acid in methylene chloride to give intermediate VIII.

As shown in Scheme 4, the piperazine intermediate VIII can be reductively alkylated with other aldehydes such as 1-trityl-4-imidazolyl-carboxaldehyde or 1-trityl-4-imidazolyl-acetaldehyde, to give products such as XVI. The trityl protecting group can be removed from XVI to give XVII, or alternatively, XVI can first be treated with an alkyl halide then subsequently deprotected to give the alkylated imidazole XVIII. Alternatively, the intermediate VIII can be acylated or sulfonylated by standard techniques.

Incorporation of a hydroxyl moiety on the sidechain carbon alpha to the amide carbonyl of compounds of the formula XVIII can be accomplished as illustrated in Scheme 5. A suitably substituted primary alcohol XIX undergoes a one carbon homologation, via a Swern oxidation, nitrile addition and hydrolysis, to provide the substituted hydroxyacetic acid XX. The trifluoromethyl ketal is formed and reacted with the previously described protected piperazine VI to provide, following deprotection, the intermediate XXI. Intermediate XXI can undergo a variety of reactions at its unsubstituted nitrogen. For example, treatment of XXI with a suitably substituted imidazolylmethyl halide to provide the instant compound XXII.

Scheme 6 illustrates incorporation of a cycloalkylalkoxycarbonyl moiety onto the piperazine nitrogen. Thus a suitably substituted alcohol XXIII is reacted with nitrophenylchloroformate to provide the intermediate XXIV, which is reacted with a suitably substituted piperazine to provide the instant compound XXV. An analogous reaction sequence alternatively provides the corresponding aminocarbonyl substitution on the piperazine nitrogen, as shown in Scheme 7.

Scheme 8 illustrates the preparation of compounds analogous to compound XXXI wherein the alcohol utilized in the first step is a suitably substituted adamantanol. The scheme also illustrates the incorporation of an indole moiety for the substiutent W in place of the preferred benzylimidazolyl moiety.

Scheme 9 illustrates the preparation of compounds analogous to compound XXXI wherein the alcohol utilized in the first step is a suitably substituted alkyl alcohol. The scheme also illustrates the incorporation of an indole moiety for the substiutent W in place of the preferred benzylimidazolyl moiety.

Scheme 10 illustrates the preparation of compounds analogous to compound XXXI wherein the alcohol utilized in the first step is a suitably substituted cyclohexanol. The scheme also illustrates the incorporation of an indole moiety for the substiutent W in place of the preferred benzylimidazolyl moiety.

5-Substituted piperazin-2-ones can be prepared as shown in Scheme 11. Reductive amination of Boc-protected amino aldehydes XXXVI (prepared from I as illustrated) gives rise to compound XXXVII. This is then reacted with bromoacetyl bromide under Schotten-Baumann conditions; ring closure is effected with a base such as sodium hydride in a polar aprotic solvent such as dimethylformamide to give XXXVIII. The carbamate protecting group is removed under acidic conditions such as trifluoroacetic acid in methylene chloride, or hydrogen chloride gas in methanol or ethyl acetate, and the resulting piperazine can then be carried on to final products as described in above.

The isomeric 3-substituted piperazin-2-ones can be prepared as described in Scheme 12. The imine formed from arylcarboxamides IXL and 2-aminoglycinal diethyl acetal (XL) can be reduced under a variety of conditions, including sodium triacetoxyborohydride in dichloroethane, to give the amine XLI. A suitably substituted amino acid I can be coupled to amine XLI under standard conditions, and the resulting amide XLII when treated with aqueous acid in tetrahydrofuran can cyclize to the unsaturated XLIII. Catalytic hydrogenation under standard conditions gives the requisite intermediate XLIV, which is elaborated to final products as described in above.

Scheme 13 illustrates the use of an optionally substituted homoserine lactone XLVII to prepare a Boc-protected piperazinone XLVIII. Intermediate XLVIII may be deprotected and reductively alkylated or acylated as illustrated in the previous Schemes. Alternatively, the hydroxyl moiety of intermediate XLVIII may be mesylated and displaced by a suitable nucleophile, such as the sodium salt of ethane thiol, to provide an intermediate IL. Intermediate XLVEI may also be oxidized to provide the carboxylic acid on intermediate L, which can be utilized from an ester or amide moiety.

Scheme 14 depicts a general method for synthesizing a key intermediate useful in the preparation of a preferred embodiment of the instant invention wherein V is phenyl and W is imidazole. A piperazine moiety can be readily added to this benzyl-imidazole intermediate as set forth in Scheme 15.

Schemes 16-20 illustrate the preparation of radiolabeled derivatives of inhibitors of GGTase-I, such as those compounds whose synthesis is described hereinabove.

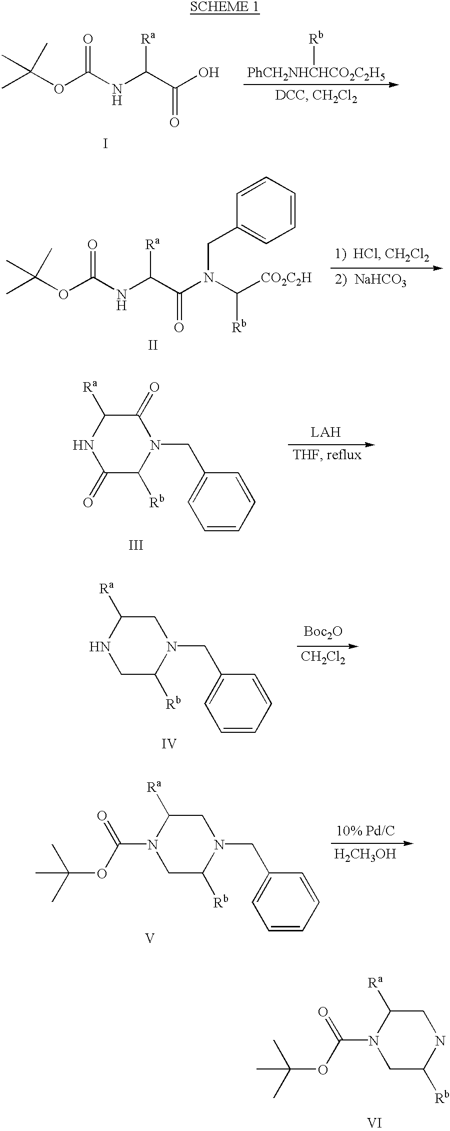

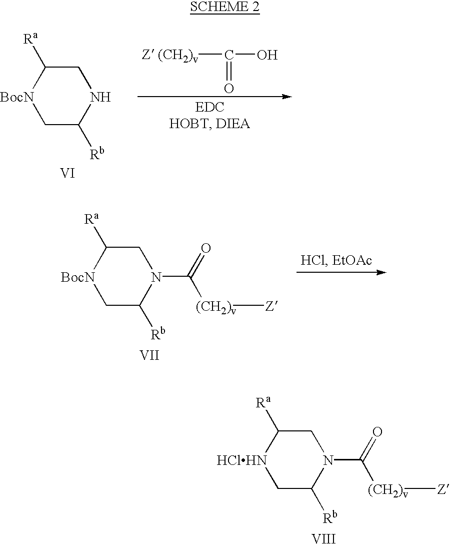

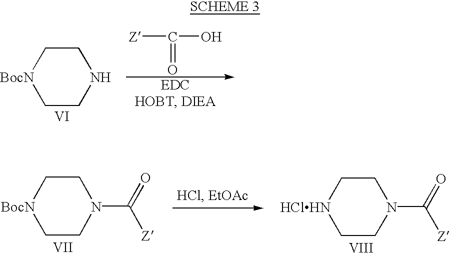

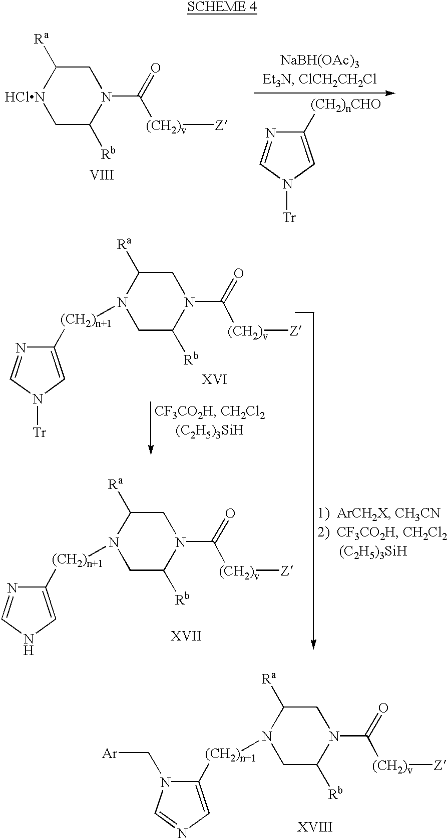

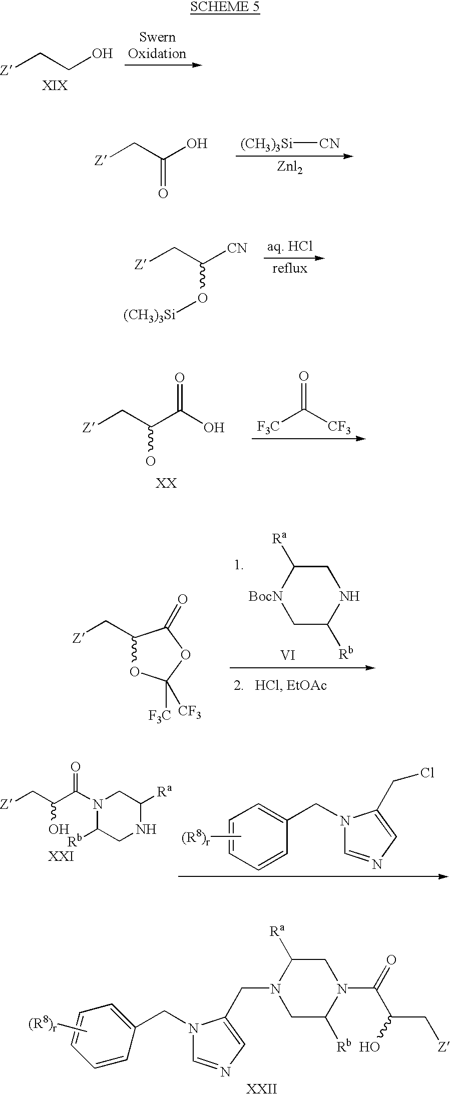

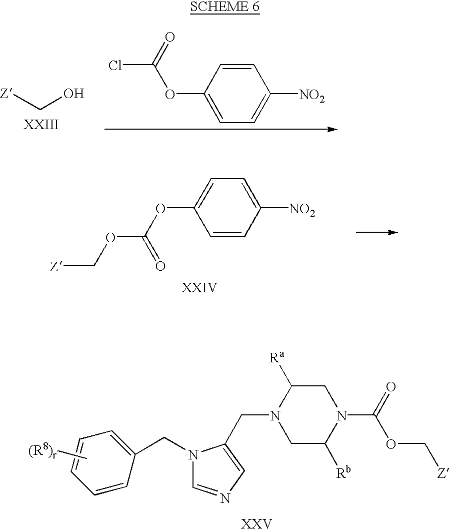

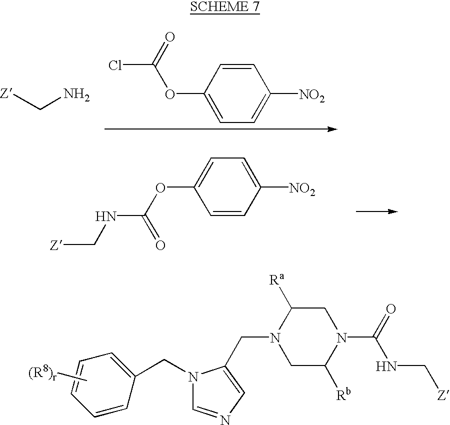

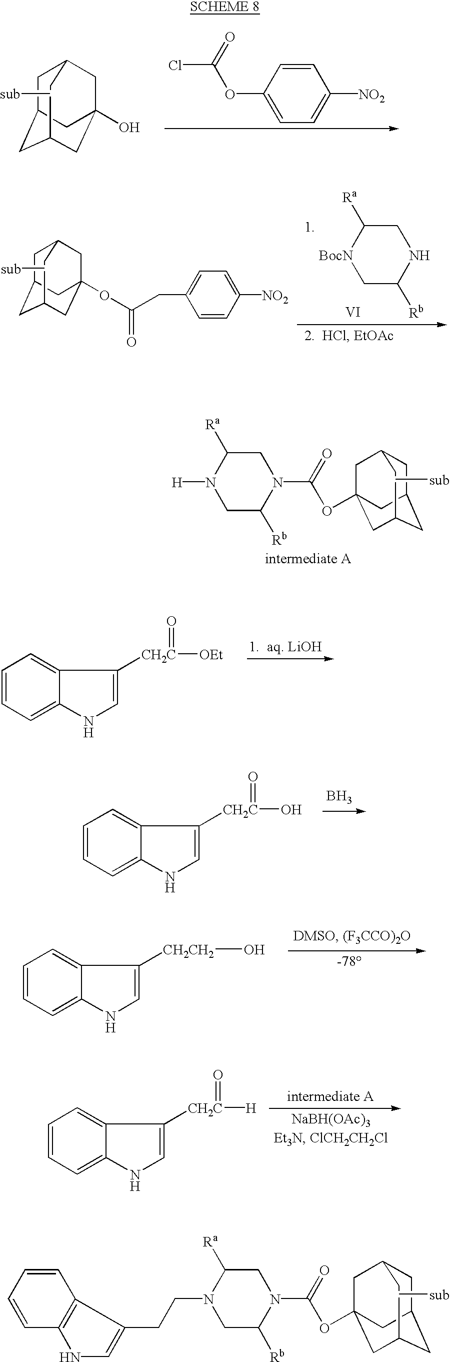

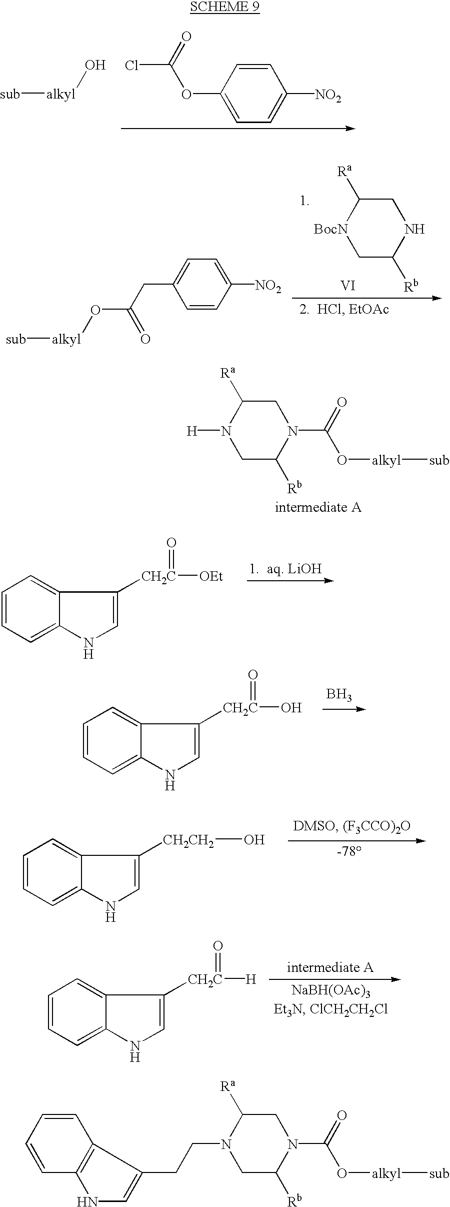

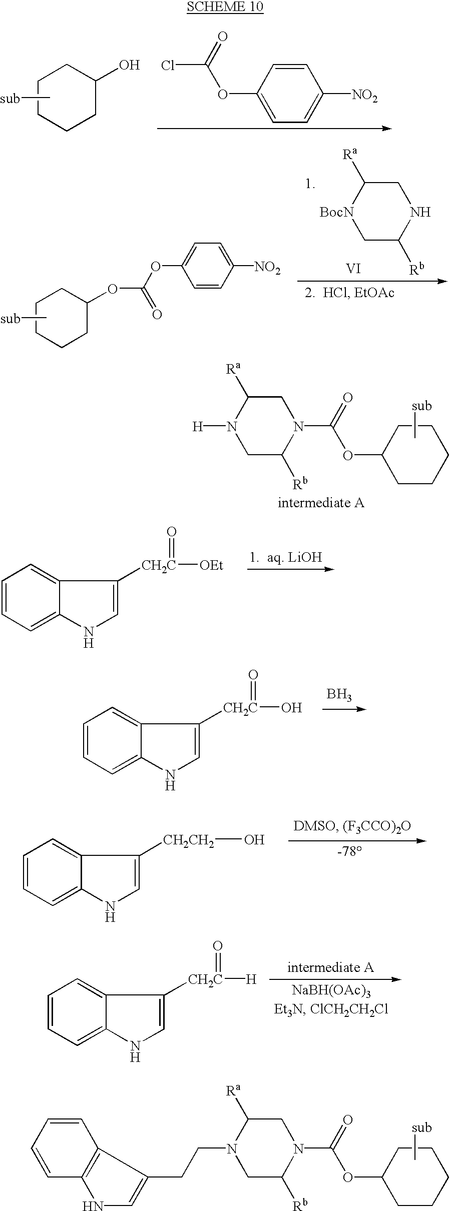

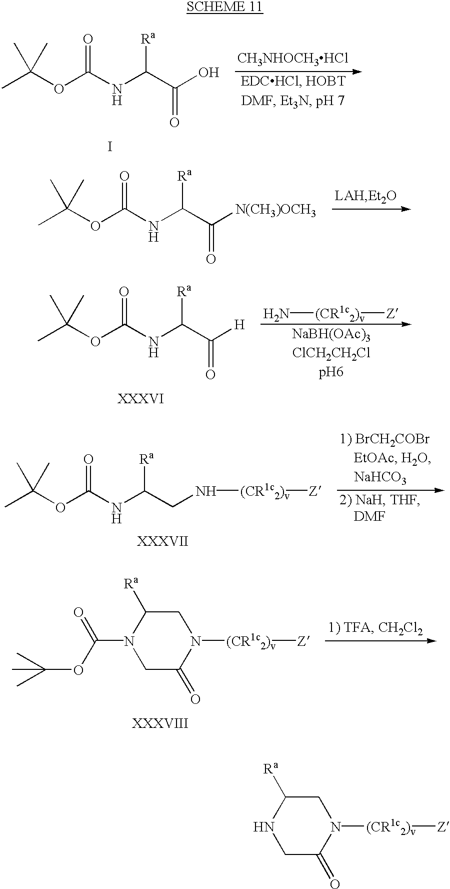

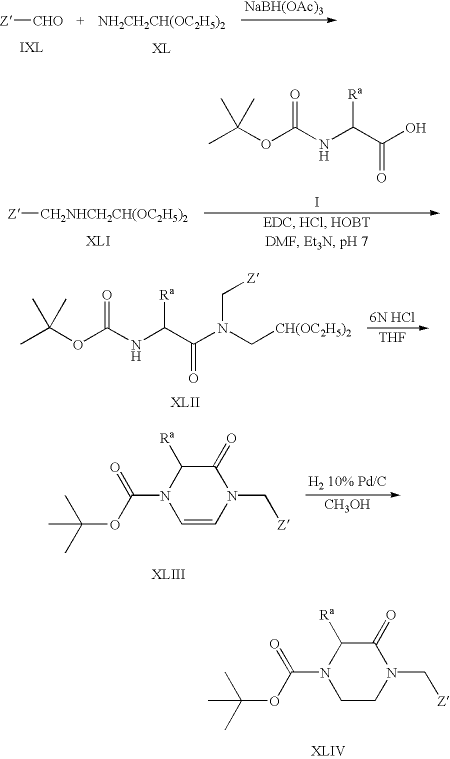

[chemical structure]

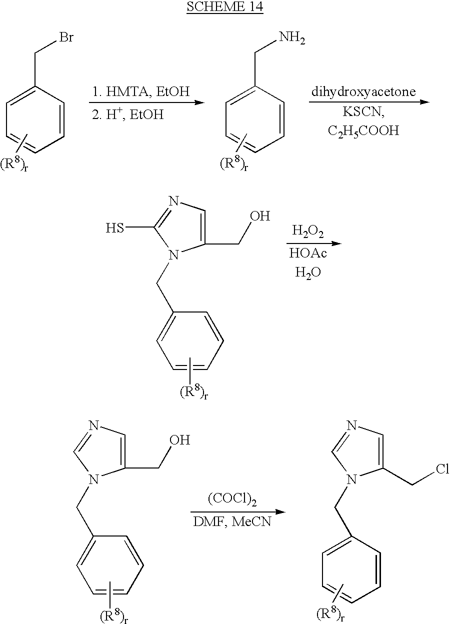

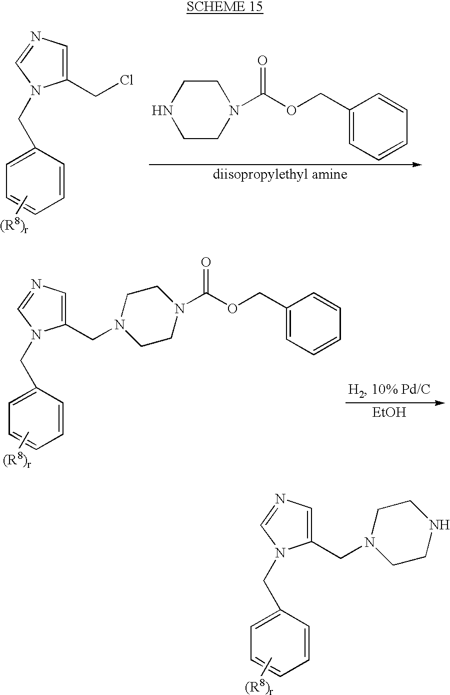

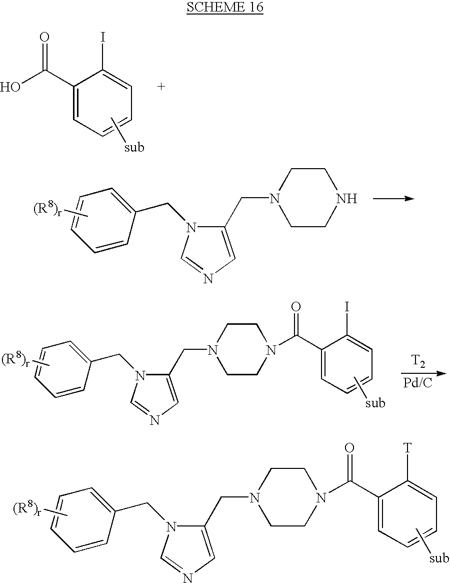

[chemical structure]

[chemical structure]

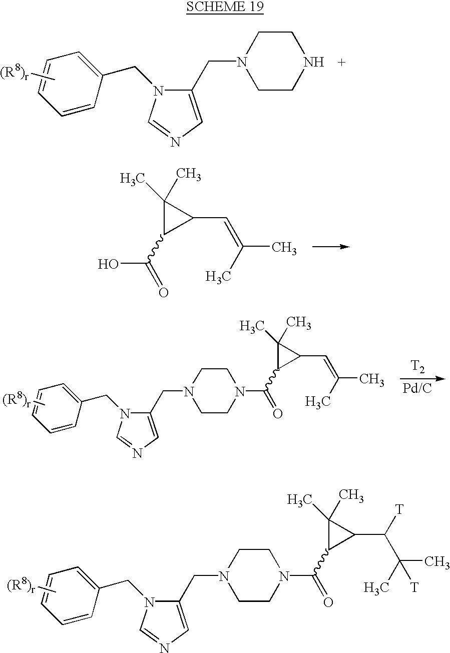

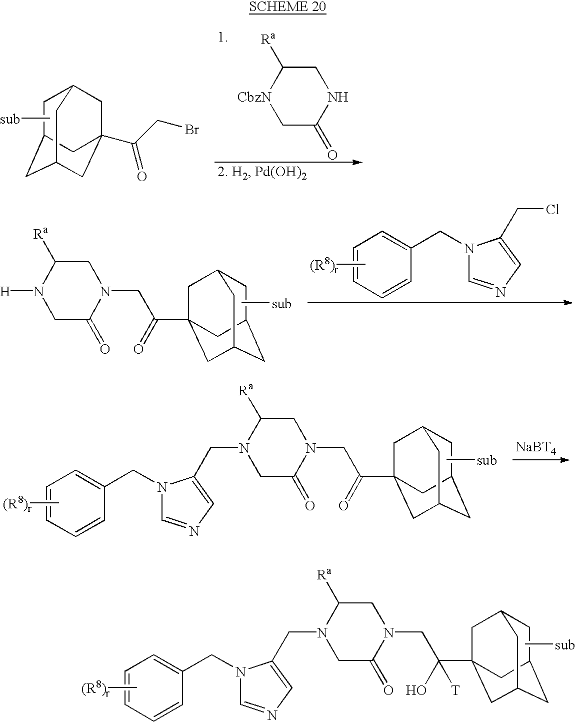

### EXAMPLES DEPICTING GENERATION OF CERTAIN COMPOUNDS

Examples provided are intended to assist in a further understanding of the invention. Particular materials employed, species and conditions are intended to be further illustrative of the invention and not a limitation on the reasonable scope thereof.

The compounds of this invention are prepared by employing reactions as shown in the examples below, in addition to other standard manipulations such as ester hydrolysis, cleavage of protecting groups, etc., as may be known in the literature or exemplified in the experimental procedures.

Geranygeranyl transferase type-I (GGTase-I) inhibitor compounds which incorporate a radionuclide may be prepared by first synthesizing an unlabeled inhibitor that optionally incorporates a iodo or bromo moiety and then exchanging a hydrogen or halogen moiety with an appropriate radionuclide using techniques well known in the art. Syntheses of unlabeled GGTase-I inhibitors have been generally described in the patent publications cited hereinabove. Syntheses of particular GGTase-I inhbitors is described below.

### Example 1

Preparation Of 1-(4-cyanobenzyl)-5-chloromethyl imidazole HCl Salt

Step 1: Preparation of 4-Cyanobenzylamine Method 1 (Hydrochloride salt):

A 72 liter vessel was charged with 190 proof ethanol (14.4 L) followed by the addition of 4-cyanobenzylbromide (2.98 kg) and HMTA (2.18 kg) at ambient temperature. The mixture was heated to about 72-75° C. over about 60 min. On warming, the solution thickens and additional ethanol (1.0 liter) was added to facilitate stirring. The batch was aged at about 72-75° C. for about 30 min.

The mixture was allowed to cool to about 20° C. over about 60 min, and HCl gas (2.20 kg) was sparged into the slurry over about 4 hours during which time the temperature rose to about 65° C. The mixture was heated to about 70-72° C. and aged for about 1 hour. The slurry was cooled to about 30° C. and ethyl acetate (22.3 L) added over about 30 min. The slurry was cooled to about −5° C. over about 40 min and aged at about −3 to about −5° C. for about 30 min. The mixture was filtered and the crystalline solid was washed with chilled ethyl acetate (3×3 L). The solid was dried under an N₂ stream for about 1 hour before charging to a 50 liter vessel containing water (5.5 L). The pH was adjusted to about 10-10.5 with 50% NaOH (4.0 kg) maintaining the internal temperature below about 30° C. At about 25° C., methylene chloride (2.8 L) was added and stirring continued for about 15 min. The layers were allowed to settle and the lower organic layer was removed. The aqueous layer was extracted with methylene chloride (2×2.2 L). The combined organic layers were dried over potassium carbonate (650 g). The carbonate was removed via filtration and the filtrate concentrated in vacuo at about 25° C. to give a free base as a yellow oil.

The oil was transferred to a 50 liter vessel with the aid of ethanol (1.8 L). Ethyl acetate (4.1 L) was added at about 25° C. The solution was cooled to about 15° C. and HCl gas (600 g) was sparged in over about 3 hours, while keeping batch temperature below about 40° C. At about 20-25° C., ethyl acetate (5.8 L) was added to the slurry, followed by cooling to about −5° C. over about 1 hour. The slurry was aged at about −5° C. for about 1 hour and the solids isolated via filtration. The cake was washed with a chilled mixture of EtOAc/EtOH (9:1 v/v) (1×3.8 L), then the cake was washed with chilled EtOAc (2×3.8 L). The solids were dried in vacuo at about 25° C. to provide the above-titled compound.

¹H NMR (250 MHz, CDCl3) δ 7.83-7.79 (d, 2H), 7.60-7.57 (d, 2H), 4.79 (s, 2H), 4.25 (s, 2H); ¹³C NMR (62.9 MHz, CDC13) δ 149.9, 139.8, 134.2, 131.2, 119.7, 113.4, 49.9, 49.5, 49.2, 48.8, 48.5, 48.2, 43.8.

Method 2 (Phosphate Salt):

A slurry of HMTA in 2.5 L EtOH was added gradually over about 30 min to about 60 min to a stirred slurry of cyanobenzylbromide in 3.5 L EtOH and maintained at about 48-53° C. with heating & cooling in a 22L neck flask (small exotherm). Then the transfer of HMTA to the reaction mixture was completed with the use of 1.0 L EtOH. The reaction mixture was heated to about 68-73° C. and aged at about 68-73° C. for about 90 min. The reaction mixture was a slurry containing a granular precipitate which quickly settled when stirring stopped.

The mixture was cooled to a temperature of about 50° C. to about 55° C. Propionic acid was added to the mixture and the mixture was heated and maintained at a temperature of about 50° C. to about 55° C. Phosphoric acid was gradually added over about 5 min to about 10 min, maintaining the reaction mixture below about 65° C. to form a precipitate-containing mixture. Then the mixture was gradually warmed to about 65° C. to about 70° C. over about 30 min and aged at about 65° C. to about 70° C. for about 30 min. The mixture was then gradually cooled to about 20-25° C. over about 1 hour and aged at about 20-25° C. for about 1 hour.

The reaction slurry was then filtered. The filter cake was washed four times with EtOH, using the following sequence, 2.5 L each time. The filter cake was then washed with water five times, using 300 mL each time. Finally, the filter cake was washed twice with MeCN (1.0 L each time) and the above identified compound was obtained.

Step 2: Preparation of 1-(4-Cyanobenzyl)-2-mercapto-5-hydroxymethylimidazole

7% water in acetonitrile (50 mL) was added to a 250 niL roundbottom flask. Next, an amine phosphate salt (12.49 g), as described in Step 1, was added to the flask. Next potassium thiocyanate (6.04 g) and dihydroxyacetone (5.61 g) was added. Lastly, propionic acid (10.0 mL) was added. Acetonitrile/water 93:7 (25 mL) was used to rinse down the sides of the flask. This mixture was then heated to 60° C., aged for about 30 minutes and seeded with 1% thioimidazole. The mixture was then aged for about 1.5 to about 2 hours at 60° C. Next, the mixture was heated to 70° C., and aged for 2 hours. The temperature of the mixture was then cooled to room temperature and was aged overnight. The thioimidazole product was obtained by vacuum filtration. The filter cake was washed four times acetonitrile (25 mL each time) until the filtrates became nearly colorless. Then the filter cake was washed three times with water (approximately 25-50 mL each time) and dried in vacuo to obtain the above-identified compound.

Step 3: Preparation of 1-(4-Cyanobenzyl)-5-Hydroxymethylimidazole

A IL flask with cooling/heating jacket and glass stirrer (Lab-Max) was charged with water (200 mL) at 25° C. The thioimidazole (90.27 g), as described in Step 2, was added, followed by acetic acid (120 mL) and water (50 mL) to form a pale pink slurry. The reaction was warmed to 40° C. over 10 minutes. Hydrogen peroxide (90.0 g) was added slowly over 2 hours by automatic pump maintaining a temperature of 35-45° C. The temperature was lowered to 25° C. and the solution aged for 1 hour.

The solution was cooled to 20° C. and quenched by slowly adding 20% aqueous Na₂SO₃ (25 mL) maintaining the temperature at less than 25° C. The solution was filtered through a bed of DARCO G-60 (9.0 g) over a bed of SolkaFlok (1.9 g) in a sintered glass funnel. The bed was washed with 25 mL of 10% acetic acid in water.

The combined filtrates were cooled to 15° C. and a 25% aqueous ammonia was added over a 30 minute period, maintaining the temperature below 25° C., to a pH of 9.3. The yellowish slurry was aged overnight at 23° C. (room temperature). The solids were isolated via vacuum filtration. The cake (100 mL wet volume) was washed with 2×250 mL 5% ammonia (25%) in water, followed by 100 mL of ethyl acetate. The wet cake was dried with vacuum/N₂ flow and the above-titled compound was obtained.

¹H NMR (250 MHz, CDC13): δ 7.84-7.72 (d, 2H), 7.31-7.28 (d, 2H), 6.85 (s, 1H), 5.34 (s, 2H), 5.14-5.11 (t, 1H), 4.30-4.28 (d, 2H), 3.35 (s, 1H).

Step 4: Preparation of 1-(4-cyanobenzyl)-5-chloromethyl imidazole HCl salt

Method 1:

1-(4-Cyanobenzyl)-5-hydroxy-methylimidazole (1.0 kg), as described in above in Step 3, was slurried with DMF (4.8 L) at 22° C. and then cooled to −5° C. Thionyl chloride (390 mL) was added dropwise over 60 min during which time the reaction temperature rose to a maximum of 9° C. The solution became nearly homogeneous before the product began to precipitate from solution. The slurry was warmed to 26° C. and aged for 1 h.

The slurry was then cooled to 5° C. and 2-propanol (120 mL) was added dropwise, followed by the addition of ethyl acetate (4.8 L). The slurry was aged at 5° C. for 1 h before the solids were isolated and washed with chilled ethyl acetate (3×1 L). The product was dried in vacuo at 40° C. overnight to provide the above-titled compound.

¹H NMR (250 MHz DMSO-d₆): δ 9.44 (s, 1H), 7.89 (d, 2H, 8.3 Hz), 7.89 (s, 1H), 7.55 (d, 2H, 8.3 Hz), 5.70 (s, 2H), 4.93 (s, 2H). ¹³C NMR (75.5 MHz DMSO-d6): δ_{c} 139.7, 137.7, 132.7, 130.1, 128.8, 120.7, 118.4, 111.2, 48.9, 33.1.

Method 2:

To an ice cold solution of dry acetonitrile (3.2 L, 15 L/Kg hydroxymethylimidazole) was added 99% oxalyl chloride (101 mL, 1.15 mol, 1.15 equiv.), followed by dry DMF (178 mL, 2.30 mol, 2.30 equiv.), at which time vigorous evolution of gas was observed. After stirring for about 5 to 10 min following the addition of DMF, solid hydroxymethylimidazole (213 g, 1.00 mol), as described above in Step 3, was added gradually. After the addition, the internal temperature was allowed to warm to a temperature of about 23° C. to about 25° C. and stirred for about 1 to 3 hours. The mixture was filtered, then washed with dry acetonitrile (400 mL displacement wash, 550 mL slurry wash, and a 400 mL displacement wash). The solid was maintained under a N₂ atmosphere during the filtration and washing to prevent hydrolysis of the chloride by adventitious H₂O . This yielded the crystalline form of the chloromethylimidazole hydrochloride.

¹H NMR (250 MHz DMSO-d6): δ 9.44 (s, 1H), 7.89 (d, 2H, 8.3 Hz), 7.89 (s, 1H), 7.55 (d, 2H, 8.3 Hz), 5.70 (s, 2H), 4.93 (s, 2H). ¹³C NMR (75.5 MHz DMSO-d6): δ_{c} 139.7, 137.7, 132.7, 130.1, 128.8, 120.7, 118.4, 111.2, 48.9, 33.1.

Method 3:

To an ice cold solution of dry DMF (178 mL, 2.30 mol, 2.30 equiv.) in dry acetonitrile (2.56 L, 12 L/Kg Hydroxymethylimidazole) was added oxalyl chloride (101 mL, 1.15 mol, 1.15 equiv). The heterogeneous mixture in the reagent vessel was then transferred to a mixture of hydroxymethylimidazole (213 g, 1.00 mol), as described above in Step 3, in dry acetonitrile (1.7 L, 8 L/Kg hydroxymethylimidazole). Additional dry acetonitrile (1.1-2.3 L, 5-11 L/Kg hydroxymethylimidazole) was added to the remaining solid Vilsmeier reagent in the reagent vessel. This, now nearly homogenous, solution was transferred to the reaction vessel at Tᵢ≦±6° C. The reaction vessel temperature was warmed to a temperature of about 23° C. to about 25° C. and stirred for about 1 to 3 hours. The mixture was then cooled to 0° C. and aged 1 h. The solid was filtered and washed with dry, ice cold acetonitrile (400 mL displacement wash, 550 mL slurry wash, and a 400 mL displacement wash). The solid was maintained under a N₂ atmosphere during the filtration and washing to prevent hydrolysis of the chloride by adventitious H₂O. This yielded the crystalline form of the chloromethylimidazole hydrochloride.

### Example 2

1-[1-(4′-Cyanobenzyl) imidazol-5-ylmethyl]piperazine

Step 1:

1-(4′-Cyanobenzyl) imidazol-5-ylmethyl piperazine-4-carboxylic acid benzyl ester

To an acetonitrile solution of 1-(4′-cyanobenzyl)-5-chloromethylimidazole (7.45 mmol) prepared as described in Example 1 and diisopropylethyl amine (22.4 mmol) was added 1-benzyl 1-piperazine carboxylate (10.4 mmol). This solution was stirred for 4.0 hour at 80° C. The product was isolated after silica column purification. ¹H-NMR (CDCl₃): 7.65 (d, 2H); 7.55 (s, 1H); 7.38 (m, 5H); 7.15 (d, 2H); 7.0 (s, 1H); 5.3 (s, 2H); 5.1 (s, 1H); 3.4 (m, 4H); 3.3 (s, 2H); 2.3 (m, 4H).

Step 2: 1-[1-(4′-Cyanobenzyl) imidazol-5-ylmethyl]piperazine

The product from Step 1 (6.17 mmol) was dissolved in absolute ethanol followed by the introduction of 10% Pd/C catalyst then hydrogen under atmospheric pressure. The catalyst was removed via filtration through filter-aid and the product was isolated by removing the solvent under reduced pressure. ¹H-NMR (CD₃OD): 7.8 (s, 1H); 7.75 (d, 2H); 7.3 (d, 2H); 6.9 (s, 1H); 5.45 (s, 2H); 3.3 (m, 4H); 2.6 (s, 2H); 2.3 (m, 4H).

### Example 3

1-(4-Cyanobenzyl)-5-[1-(2-oxo-2-(adamant-1-yl)ethyl)-2-oxo-piperazin-4-yl-methyl]imidazole

Step 1: 4-Benzyloxycarbonyl-[1-(2-oxo-2-(adamant-1-yl))ethyl]piperazin-2-one

To a round bottomed flask were added 4-benzyloxycarbonylpiperazin-2-one (234.26 mg, 1.0 mmol) along with N,N-dimethylforamide (5.0 ml), and sodium hydride (73.0 mg (60%), 1.0 mmol). After the evolution of hydrogen had ceased the reaction was allowed to stir for an additional 30 minutes. To this was added 1 adamantyl bromomethyl ketone (257.18 mg, 1.0 mmol). The reaction stirred at Rt. for 18hrs. The reaction was poured into water (25 ml) and extracted with ethylacetate (2×25 ml). The ethylacetate layer was washed with brine and dried (MgSO₄). Solvent removal yielded 4-benzyloxycarbonyl-[1-(2-oxo-2-(adamant-1-yl)ethyl]piperazin-2-one. 400 Mhz H¹ NMR (CDCl₃): 1.68-1.91(m,12H), 2.06 (br s,3H), 3.32(t,2H), 3.77(t,2H), 4.20(s,2H), 4.34(s,2H), 5.16(s,2H), 7.36(m,5H). The material was used with out further purification.

Step 2: 1-[1-(2-oxo-2-(adamant-1-yl))ethyl]piperazin-2-one

4-Benzyloxycarbonyl-[1-(2-oxo-2-(adamant-1-yl))ethyl]piperazin-2-one (389.2 mg, 0.9 mmol) was placed it a parr flask along with palladium hydroxide on carbon (50 mg, 20 wt. %), Ethanol (75.Oml), Water (5.0 ml), and 2 drops of conc. HCl. The flask was placed on the Parr apparatus and charged with 50 psi of hydrogen. The reaction was allowed to shake for four hours. The reaction was filtered through a celite column and the solvents removed under high vacuum. This resulted in 1-[1-(2-oxo-2-(adamant-1-yl))ethyl]piperazin-2-one. 400 Mhz H¹ NMR (CD₃OD): 1.74-1.83(m,6H), 1.91 (br s,6H), 2.05(br s,3H), 3.58(s,4H), 3.90(s,2H), 4.50(s,2H). The material was used with out further purification.

Step 3: 1-(4-Cyanobenzyl)-5-[1-(2-oxo-2-(adamant-1-yl)ethyl)-2-oxo-piperazin-4-yl-methyl]imidazole

1-[1-(2-Oxo-2-(adamant-1-yl))ethyl]piperazin-2-one (253.4 mg, 0.81 mmol) was placed into a round bottomed flask and diisopropylethylamine (0.71 ml), acetonitrile (20.0 ml) and 1-(4-cyanobenzyl-5-chloromethyl)imidazole hydrochloride (prepared as described in Example 1) (217.2 mg, 0.81 mmol). The reaction was heated at reflux for 3 hours. The excess acetonitrile was removed under vacuum and the residue dissolved into ethylacetate (25.Oml). The ethylacetate layer was washed with water and the aqueous layer back extracted with additional ethylacetate(15.Oml). The combined ethylacetate extracts were dried (MgSO₄). Solvent removal yielded 249.0 mg (65%) of a yellow foam. The foam was dissolved in to 1.ON HCl and washed with hexane/ethylacetate 25 ml (75/25). The aqueous layer was then basified with ammonium hydroxide and the aqueous layer reextracted with ethylacetate. The organic layer was again dried (MgSO₄) and solvent removed. The residue was treated with a dioxane/methanol/HCl solution which gave, after lyophlizing, 1-(4-Cyanobenzyl-5-[1-(2-oxo-2-(adamant-1-yl)ethyl)-2-oxo-piperazin-4-yl-methyl]imidazole dihydrochloride. 400 Mhz H¹ NMR (CDCl₃): 1.55-1.75(m,12H), 2.01(br s,3H), 2.40-2.61(m,2H), 3.06-3.19(m,5H), 3.32(s,2H), 3.71-3.75(d,1H), 5.29(s,2H), 7.02(s,1H), 7.14(d,2H), 7.58(s,1H), 7.64(d,2H)

### Example 4

R/S 1-(4-Cyanobenzyl)-5-[1-(2-hydroxy-2-(adamant-1-yl)ethyl)-2-oxo-piperazin-4-yl-methyl]imidazole (Compound A)

A solution of 201 mg (.4 mmol) of 1-(4-cyanobenzyl-5-[1-(2-oxo-2-(adamant-1-yl)ethyl)-2-oxo-piperazin-4-yl-methyl]-imidazole (prepared as described in Example 3), 138.0 mg (1.0 mmol) sodium carbonate in methanol 10 ml was treated with sodium borohydride (22.6 mg, 0.60 mmol) at 0° C. The reaction was allowed to warm to Rt. The reaction was diluted with water/Brine and the reaction extracted with ethyl acetate. The ethylacetate layer was washed with water/brine and dried (MgSO₄). Solvent removal yielded 189.0 mg of a solid. The solid was flashed through a small silica column eluting with methanol/chloroform (10/90). This resulted in R/S 1-(4-Cyanobenzyl-5-[1-(2-hydroxy-2-(adamant-1-yl)ethyl)-2-oxo-piperazin-4-yl-methyl]imidazole. 400 Mhz H¹ NMR (CDCl₃): 1.55-1.75(m,12H), 2.01(br s,3H), 2.40-2.61(m,2H), 3.06-3.19(m,5H), 3.32(s,2H), 3.71-3.75(d,1H), 5.29(s,2H), 702(s,1H), 7.14(d,2H), 7.58(s,1H), 7.64(d,2H). High Res. FAB MS: Theoretical Mass 474.2864, Measured Mass 474.2864 (C₂₈H₃₅N₅O₂+H⁺).

### Example 5

Preparation of Amides from Acids

To each of twelve previously tared screw cap test tubes was added approximately 0.12 mmol of one of the acids. After determining this weight by difference, a DCM solution of 2,3-dimethyl-2-fluoro-pyridinium tosylate (1.2 equivalents vs. acid, 218 mg/mL) was added to each tube and then a DCM solution of triethyl amine (1.2 equivalents vs. acid, 250)L/μmL DCM) was added. These solutions were stirred for 15 minutes and then a solution of 1-(4′-cyanobenzyl) imidazol-5-ylmethyl piperazine, prepared as described in Example 2 (0.1 mmol in DMF) and DIEA (0.2 mmol) in DCM (0.5 mL) for a total volume of 0.615 mL was added to each tube and then stirred for 5 hours. Each tube was washed with 2×1 mL of 1% trifluoroacetic acid. The DCM layers were removed and then transferred to new pre-tared test tubes and then rotary speed evaporated to a pellet. The resulting pellet was dissolved in 2.0 mL of DMSO and submitted for LC and HI-RES Mass spec analysis. The completeness of reaction was estimated by comparing the AUC of each reaction against an independently prepared standard of known concentration that was also prepared in the library run.

The following compounds were prepared using this procedure:

[chemical structure]

1-[1-(4′-Cyanobenzyl)imidazol-5-ylmethyl]4-(2,2-dicyclohexyl)acetyl piperazine Hi-Res MS: calc: 488.3396 found: 488.3384

[chemical structure]

1-[1-(4′-Cyanobenzyl)imidazol-5-ylmethyl]piperazine-4-(2,2-dimethyl-3-isobutenylcyclopropane)carboxamide Hi-Res MS: calc: 432.2779 found: 432.2758

[chemical structure]

trans-1-[1-(4-cyanophenyl)methylimidazol-5-ylmethyl]-4-[t-butyloxycarbonyl)aminomethyl-4-cyclohexanecarbonyl Hi-Res MS: calc: 521.3235 found: 521.3234

[chemical structure]

1-[1-(4-cyanophenyl)methylimidazol-5-ylmethyl]-4-(2,2-dimethyl-1-cyclopropanecarbonyl)piperazine Hi-Res MS: calc: 378.2288 found: 378.23

[chemical structure]

Hi-ResMS: calc: 511.2810 found: 511.2816

### Example 6

Preparation of 1-[1-(4′-Cyanobenzyl) imidazol-5-ylmethyl] piperazine-4-carboxylic acid-(2,6-dimethoxy)benzyl ester

Step 1: Preparation of 2,6-Dimethoxybenzyloxy-(4-nitropheny)carbonate

A THF:acetonitrile (7:1, 2 mL) solution of 4-nitrophenyl chloroformate (0.61 mmol) was added to a THF:acetonitrile (7:1, 2 mL) solution of 2,6-dimethoxybenzyl alcohol (0.103 g, 0.61 mmol) at 25° C. and then stirred for 0.25 hour. Pyridine (0.61 mmol) was then added dropwise over 1 minute. Stirring was continued for 2 hours at 25° C. and then the reaction was diluted with ethyl acetate and washed with water, a saturated sodium chloride solution, dried with sodium sulfate and then evaporated to give the title compound.

¹H-NMR (CDCl₃): δ 8.3 (d, 2H); 7.4 (d, 2H); 7.3 (t, 1H); 6.6 (d, 2H); 5.5 (s, 2H); 3.9 (d, 6H).

Step 2: Preparation of 1-[1-(4′-Cyanobenzyl) imidazol-5-ylmethyl] piperazine-4-carboxylic acid-(2,6-dimethoxy)benzyl ester

To a DMF solution (2 ml) of 1-(4′-cyanobenzyl) imidazol-5-ylmethyl piperazine (0.067 g, 0.24 mmol) (prepared as described in Example 2, Step 2) and diisopropylethyl amine (0.48 mmol) was added the product from Step 1 (0.084 g, 0.25 mmol) with stirring for 12 hours at 25° C. The title compound was isolated after purification on a preparative hplc via lyophilization.

FAB-MS: calc: 475.5, found: 476.3. ¹H-NMR (CD₃OD): δ 8.2 (s, 1H); 7.8 (d, 2H); 7.3 (m, 3H); 7.1 (s, 1H); 6.6 (d, 2H); 5.5 (s, 2H); 5.1 (s, 2H); 3.8 (s, 6H); 3.4 (s, 2H); 3.2 (m, 4H); 2.3 (m, 4H).

### Example 7

Preparation of 1-[1-(4′-cyanobenzyl) imidazol-5-ylmethyl]-4-[1-(2,6-dimethylbenzyloxycarbonyl] piperazine bis trifloroacetate salt

A solution of 250 mg (1.84 mmol) of 2,5-dimethylbenzyl alcohol and 409 mg (2.03 mmol) of p-nitrophenylchloroformate in 5 ml of 7:1 THF/acetonitrile under an argon atmosphere was treated with 164 ml (2.03 mmol) of pyridine, and the resulting suspension was stirred vigorously at room temp. for 18h. The reaction was concentrated in vacuo to give a clear oil. The oil was dissolved in a minimum of chloroform and was chromatographed over silica gel with 9:1 hexanes/ethyl acetate as eluant. Product fractions were combined and concentrated in vacuo to give the carbonate intermediate as an off-white solid.

400 Mhz H¹ NMR(CDCl₃): δ 2.44 (d, 6H), 5.44 (s, 2H), 7.09 (d, 2H), 7.20 (t, 1H), 7.40 (d, 2H), 8.29 (d, 2H).

A solution of 219 mg (0.71 mmol) of the above prepared carbonate intermediate, 200 mg (0.71 mmol) of 1-(4′-Cyanobenzyl) imidazol-5-ylmethyl piperazine (prepared as described in Example 2, Step 2) and 247 ml (1.42 mmol) of DIEA in 2 ml of methylene chloride was stirred at room temp. for 18 h. The reaction was concentrated in vacuo to a yellow oil. The oil was purified by reversed phase preparatory LC, and the pure fractions combined and concentrated to remove volatiles. Lyophilization of the aqueous residue provided the bis trifluoroacetic acid salt of the desired product as an amorphous fluffy white powder. FAB MS: M+=444.2.400 Mhz H¹ NMR(CDCl₃): δ 2.39 (s, 6H), 2.65 (br s, 4H), 3.58 (br s, 4H), 3.67 (s, 2H), 5.22 (s, 2H), 5.57 (s, 2H), 7.04 (d, 2H), 7.18 (t, 1H), 7.26 (d, 2H), 7.54 (s, 1H), 7.72 (d, 2H), 8.80 (s, 1H).

### Example 8

Preparation of 1-[1-(4′-cyanobenzyl) imidazol-5-ylmethyl]-4-[1-(2-methoxy-5-chlorobenzyloxycarbonyl] piperazine

In a manner identical to that described above in Example 7, from 220 mg (0.64 mmol) of (2-methoxy-5-chlorobenzyl)-(4-nitrophenyl) carbonate (prepared as described above in Example 10 from p-nitrophenylchloroformate and 2-methoxy-5-chlorobenzyl alcohol) and 180 mg (0.64 mmol) of 1-(4′-Cyanobenzyl) imidazol-5-ylmethyl piperazine (prepared as described in Example 2, Step 2) was obtained the bis trifluoroacetic acid salt of the title compound as an amorphous fluffy white powder.

Fab MS: M+=480.18. 400 Mhz H¹ NMR(CDCl₃): δ 2.62 (br s, 4H), 3.59 (br s, 2H+4H), 3.92 (s, 3H), 5.16 (s, 2H), 5.59 (s, 2H), 6.81 (d, 1H), 7.18-7.36 (complex, 2H+2H), 7.52 (s, 1H), 7.76 (d, 2H), 8.84 (s, 1H).

Method of Radiolabeling Compounds

Unless otherwise mentioned, solvents and reagents were purchased from either Aldrich-Sigma or Fisher Scientific. High performance liquid chromatography (HPLC) analyses of radioinert compounds were performed on a system consisting of a Waters 600E gradient pump, a Rheodyne injector. The UV detection results from a Waters 991 diode array detector and the on-line detection results from a Beckman 171HPLC radioactivity detector were collected and processed. Preparative HPLC purifications were performed on either an Alltech C₁₈ Econosil semi-preparative column (10 mm×25 cm) or a Vydac C₁₈ column (4×25 cm). Sample radioactivities were determined on a LKB Wallac 1410 scintillation counter and UV measurements were performed on a HP-8452A diode array spectrophotometer.

[³H]-R/S 1-(4-Cyanobenzyl)-5-[1-(2-hydroxy-2-(adamant-1-yl)ethyl)-2-oxo-piperazin-4-vl-methyl]imidazole ([³H]-Compound A)

Thus, a suspension, which was formed by adding 1.3 mg of Na₂CO₃ into a solution of 1-(4-Cyanobenzyl)-5-[1-(2-oxo-2-(adamant-1-yl)ethyl)-2-oxo-piperazin-4-yl-methyl]imidazole, prepared as described in Example 3 (6 mg) in 250 μL of dry MeOH, was introduced into a vial containing 1 Ci of NaBT₄. The resulting mixture was stirred at room temperature for 35 min. Brine (0.75 mL) was added. The mixture was then extracted with EtOAc (5×5 mL). The combined organic layers were dried (MgSO₄) and solvents removed in vacuo to give the product mixture. Labile tritium was removed by co-evaporation with 10 mL ethanol.

A fraction of the mixture was concentrated in vacuo and first repeatedly purified with an Alltech semiprep HPLC column (20 to 40% CH₃CN/H₂O, both containing 0.1% TFA, 1.5-2 mL/min). The fraction containing the desired product was collected and further repeatedly purified with a Vydac C18 column (30-100% CH3CN/H20, both containing 0.1% TFA, 1 mL/min). Thus, a pure product fraction of [³H]-Compound A was obtained, the HPLC analysis of which (Vydac C18, 40% CH₃CN/H₂O, both containing 0.1% TFA, 1 mL/min) revealed >99% of the desired radiotracer. The specific activity of the radiotracer was found to be >13 Ci/mmol, based on the radioactivity and the mass, determined by UV analysis (254 nm) against a standard curve established with a non-radioactive standard of Compound A, prepared as described in Example 4.

Alternatively, a mixture, which is formed by adding 1.3 mg of Na₂CO₃ into a solution of 1-(4-Cyanobenzyl)-5-[1-(2-oxo-2-(adamant-1-yl)ethyl)-2-oxo-piperazin-4-yl-methyl]imidazole, prepared as described in Example 3 (6 mg) in 250 μL of dry EtOH, is introduced into a vial containing 1 Ci of NaBT₄. The resulting mixture is stirred at room temperature for 35 min. Brine (0.75 mL) is added. The mixture is then extracted with EtOAc (5×5 mL). The combined organic layers is dried (MgSO₄) and solvents removed in vacuo to give the product mixture. Labile tritium is removed by co-evaporation with 10 mL ethanol.

The mixture is repeatedly purified with an Alltech semiprep HPLC column (20 to 40% CH₃CN/H₂O, both containing 0.1% TFA, 1.5-2 mL/min). The fraction(s) containing the desired product is collected and further repeatedly purified with a Vydac C18 column (30-100% CH₃CN/H₂O, both containing 0.1% TFA, 1 mL/min). Analysis of purity and specific radioactivity is performed as described above.

Biological Assay

Cell Radiotracer Assay of Geranylgeranyl Transferase Inhibitor (CRAGGTI)

The instant invention is also directed to an assay that measures the competition between a GGTase-I inhibitor test compound and a radiolabeled GGTase-I inhibitor for binding to GGTase-I binding sites in living cells. Such an assay for example would comprise the steps of:

a) culturing monolayers of cells;

b) exposing a monolayer of cells to growth media containing the radiolabeled GGTase-I inhibitor in the presence or absence of the test compound;

c) washing the cells;

d) counting the radiation emitted by the cells; and

e) comparing the radiation emitted by cells exposed to the radiolabeled GGTase-I inhibitor and the test compound to the radiation emitted by cells exposed to only the radiolabeled GGTase-I inhibitor.

This invention is a competitive radioligand binding assay for GGTase-I that can be used for the determination of the relative inhibitory activity of compounds for the GGTase-I enzyme in living cells. The assay uses a radiolabeled GGTase-I inhibitor such as [³H] Compound A, and measures the amount of binding of this radioligand to cells. By incubating this radioligand with increasing concentrations of unlabeled Compound A, one can determine the amount of specifically bound [³H] Compound A in a population of cells, which presumably represents a specific high affinity interaction with GGTase-I. Likewise, by determining the concentrations of a test compounds required to reduce this specific high affinity interaction of [³H] Compound A in cells, one can determine relative IC₅₀s of the test compounds.

Briefly, the method involves growing a cell line in 24-well cell culture plates as an adherent population of cells until the cell culture is nearly confluent. First, the concentration of radioligand [³H] Compound A required to achieve half-maximal specific binding to cells is determined by adding [³H] Compound A at varying concentrations to 1 ml of cell culture media, and incubating this mixture with the cells for a defined period of time, such as two hours. To determine the level of non-specific binding of the radioligand, 1000-fold molar excess of unlabeled Compound A is added to some of the wells. After incubation, the unbound [³H] Compound A is removed from the cells by vacuum aspiration. The cell layer is quickly rinsed twice with 1 ml PBS (saline) to wash away unbound radiotracer. The cell layer is then solubilized with 1 ml of Solvable® (a tissue solubilizer from Packard) by incubating the cells at 50° C. for 15 min. The cell lysate is mixed with 10 ml scintillation fluid and held overnight at room temperature prior to scintillation counting. Having determined the concentration of [³H] Compound A that achieves half maximal binding to cells (apparent Kd, ˜0.3 nM [³H] Compound A in the H-ras transformed Rat1 fibroblast cell line), one can determine the relative IC₅₀s of test compounds by incubating varying concentrations of test compounds with the apparent Kd concentration of [³H] Compound A, and performing the cell incubations, washes, and solubilization as described.

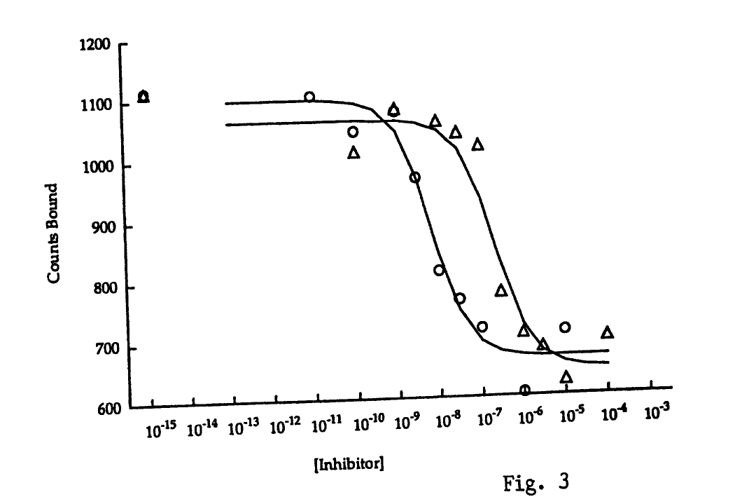

Representative [[³H] Compound A binding data and representative competition curve with unlabeled Compound A and 1-(3-Chlorophenyl)-4-[1-(4-cyanobenzyl)-5-imidazolylmethyl]-2-piperazinone are in FIG. 3.

What is claimed is:

1. An assay for determining the affinity of a geranylgeranyl transferase type I inhibitor test compound for geranylgeranyl transferase type I binding sites in cultured cells which comprises measuring the competition between the test compound and a radiolabeled geranylgeranyl transferase type I inhibitor for the geranylgeranyl transferase type I binding sites.

2. The assay of claim 1 which comprises the steps of:
a) exposing a monolayer of cells to growth media containing the radiolabeled geranylgeranyl transferase type I inhibitor in the presence or absence of the test compound;
b) washing the cells;
C) counting the radiation emitted by the cells; and
d) comparing the radiation emitted by cells exposed to the radiolabeled GGTase-I inhibitor and the test compound to the radiation emitted by cells exposed to only the radiolabeled GGTase-I inhibitor.

3. The assay of claim 2 wherein the radiolabeled GGTase-I is a compound selected from:
i) a compound of the formula A: 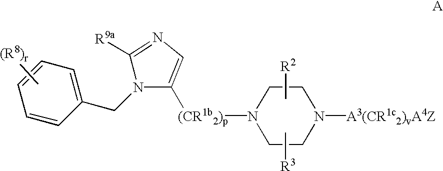
wherein:
R^{1b} is independently selected from:
a) hydrogen, βb) aryl, heterocycle, cycloalkyl, R¹⁰O—, —N(R¹⁰)₂ or C₂-C₆ alkenyl, βc) C₁-C₆ alkyl unsubstituted or substituted by aryl, heterocycle, cycloalkyl, alkenyl, R¹⁰O—, or —N(R¹⁰)₂;
R^{1c} is independently selected from:
a) hydrogen, βb) R¹⁰O—, R¹⁰C(O)NR¹⁰—, (R¹⁰)₂NC(O)—, R¹⁰₂N—C(NR¹⁰)—, CN, NO₂, R¹⁰ C(O)—, N₃, —N(R¹⁰)₂ or R¹¹OC(O)NR¹⁰—,
c) unsubstituted or substituted C₁-C₆ alkyl wherein the substitutent on the substituted C₁-C₆ alkyl is selected from R¹⁰O—, R¹⁰C.(O)NR¹⁰—, (R¹⁰)₂NC(O)—, R¹⁰₂N—C(NR¹⁰)—, CN, R¹⁰C(O)—, N₃, —N(R¹⁰)₂ and R¹¹OC(O)-NR¹⁰—;
R³ is selected from H and CH₃;
NR ⁶R⁷
R² is selected from 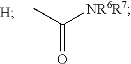
or C₁₋₅ alkyl, unbranched or branched, unsubstituted or substituted with one or more of:
1) aryl,
2) heterocycle,
3) OR⁶,
4) SR^{6a}, SO₂R^{6a}, or
5) 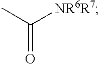
and R² and R³ are optionally attached to the same carbon atom;
R⁶ and R⁷ are independently selected from: H; C₁-4 alkyl, C₃-6 cycloalkyl, aryl, heterocycle, unsubstituted or substituted with:
a) C₁₋₄ alkoxy,
b) halogen, or
c) aryl or heterocycle;
R^{6a} is selected from: C₁₋₄ alkyl or C₃₋₆ cycloalkyl, unsubstituted or substituted with:
a) C₁₋₄ alkoxy,
b) halogen, or
c) aryl or heterocycle;
R⁸ is independently selected from:
a) hydrogen,
b) C₁-C₆ alkyl, C₂-C₆ alkenyl, C₂-C₆ alkynyl, C₁-C₆ perfluoroalkyl, F, Cl, R¹⁰O—, R¹⁰C(O)NR¹⁰—, CN, NO₂, (R¹⁰)₂N—C(NR¹⁰)—, R¹⁰C(O)—, —N(R¹⁰)₂, or R¹¹OC(O)NR¹⁰—, and
c) C₁-C₆ alkyl substituted by C₁-C₆ perfluoroalkyl, R¹⁰O—, R¹⁰C(O)NR¹⁰—, (R¹⁰)₂N—C(NR¹⁰)—, R¹⁰C(O)—, —N(R¹⁰)₂, or R¹¹C(O)NR¹⁰—;
R^{9a} is hydrogen or methyl;
R¹⁰ is independently selected from hydrogen, C₁-C₆ alkyl, benzyl and aryl;
R¹¹ is independently selected from C₁-C₆ alkyl and aryl;
A³ is selected from: —C(O)—, —C(O)NR¹⁰— or —C(O)O—;
A⁴ is selected from: bond and —O—;
Z is substituted C₅-C₇ cycloalkyl, wherein the substituted C₅-C₇ cycloalkyl is substituted with one or more C₁₋₄ alkyl moieties and is optionally substituted with one or two moieties selected from the following:
a) C₁₋₄ alkoxy,
b) NR⁶R⁷,
c) C₃₋₆ cycloalkyl,
d) —NR⁶C(O)R⁷,
e) HO,
f) —S(O)ₘR^{6a},
g) halogen, or
h) perfluoroalkyl;
m is 0, 1 or 2;
p is 1, 2 or 3;
r is 0 to 5, and
v is 0, 1, 2 or 3;
and wherein at least one radionuclide or ³H-methyl is present in the molecule;
ii) a compound of the formula B: 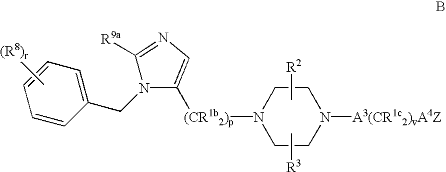
wherein:
R^{1b}, R^{1c}, R², R³, R⁶, R⁷, R^{6a}, R⁸, R^{9a}, R¹⁰, R¹¹, A³, A⁴, m, p, r and v are as defined above for the compound of the formula A;
Z is an unsubstituted or substituted C₇-C₁₀ multicyclic alkyl ring, wherein the substituted C₇-C₁₀ multicyclic alkyl ring is substituted with one or two moieties selected from the following:
a) C1 4 alkoxy,
b) NR⁶R⁷,
c) C₃₋₆ cycloalkyl,
d) —NR⁶C(O)R⁷,
e) HO,
f) —S(O)ₘR^{6a},
g) halogen,
h) perfluoroalkyl, and
i) C₁₋₄ alkyl;
C₇-C₁₀ multicyclic alkyl ring is selected from: 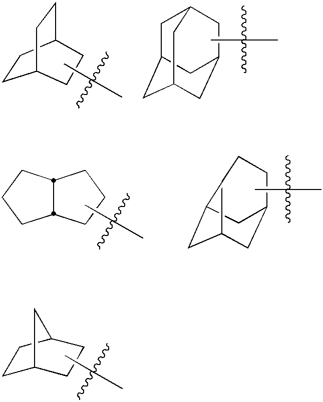
and wherein at least one radionuclide or H-methyl is present in the molecule;
iii) a compound of the formula C: 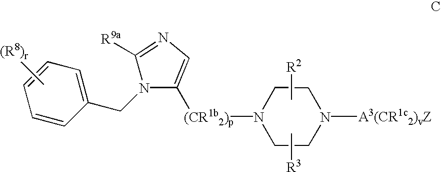
wherein:
R^{1c}, R³, R⁶, R⁷, R^{6a} , R⁸, R¹⁰, R¹¹, m, p and r are as defined above for the compound of the form ula A;
R^{1b} is independently selected from:
a) hydrogen,
b) aryl, heterocycle, cycloalkyl, CN, R¹⁰O—, R¹⁰NC(O)—,
—N(R¹⁰)₂ or C₂-C₆ alkenyl,
c) C₁-C6 alkyl unsubstituted or substituted by aryl, heterocycle, cycloalkyl, alkenyl, R¹⁰O—, or —N(R¹⁰)₂;
R² is selected from 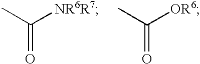
or C₁₋₅ alkyl, unbranched or branched, unsubstituted or substituted with one or more of:
1) aryl,
2) heterocycle,
3) OR⁶,
4) SR^{6a}, SO₂R^{6a}, or
5) 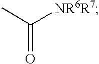
and R² and R³ are optionally attached to the same carbon atom;
R^{9a} is hydrogen, C₁-C₆ alkyl or chloro;
A³ is selected from: —C(O)—, —C(O)NR¹⁰—, —C(O)O— and S(O)ₘ;
Z is unsubstituted or substituted phenyl, unsubstituted or substituted napthyl, unsubstituted or substituted pyridyl, unsubstituted or substituted quinoline or unsubstituted or substituted 1,2 methylenedioxybenzene; and
v is 0, 1, 2 or 3; provided that v is not 0 if A³ is —C(O)— or S(O)ₘ;
and wherein at least one radionuclide or ³H-methyl is present in the molecule;
iv) a compound of the formula D: 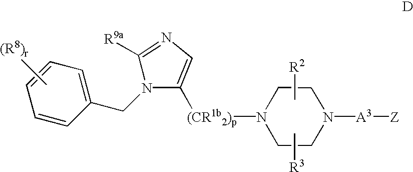
wherein:
R^{1b}, R², R³, R⁶, R⁷, R^{6a}, R⁸, R^{9a}, R¹⁰, R¹¹, A³, m, p, r and v are as defined above for the compound of the formula A;
Z is unsubstituted or substituted C₅-C₁₀ alkyl, wherein the substituted C₅-C₁₀ alkyl is substituted with one or two moieties selected from the following:
a) C₁₋₄ alkoxy,
b) NR⁶R⁷,
c) C₃₋₆ cycloalkyl,
d) —NR⁶C(O)R⁷,
e) —OR¹⁰,
f) —S(O)ₘR^{6a},
g) halogen, or
h) perfluoroalkyl;
and wherein at least one radionuclide or ³H-methyl is present in the molecule;
v) a compound of the formula E: 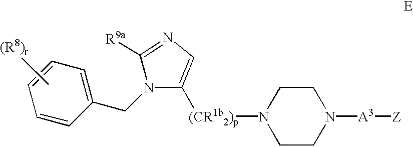
wherein:
R^{1b}, R⁸, R¹¹, p and r are as defined above for the compound of the formula A;
R^{9a} is hydrogen, C₁-C₆ alkyl or chloro;
R¹⁰ is independently selected from hydrogen, C₁-C₆ alkyl, benzyl and aryl;
A³ is —C(O)—;
Z is unsubstituted or substituted phenyl, unsubstituted or substituted napthyl, unsubstituted or substituted pyridyl, unsubstituted or substituted 2,3-dihydrobenzofuran, unsubstituted or substituted quinoline or unsubstituted or substituted isoquinoline;
and wherein at least one radionuclide or ³H-methyl is present in the molecule;
or a pharmaceutically acceptable salt thereof.

4. The assay of claim 2 wherein the radiolabeled GGTase-I inhibitor is selected from a radiolabeled derivative of a compound selected from the group of compounds comprising:
1-[1-(4′-Cyanobenzyl) imidazol-5-ylmethyl]piperazine-4-carboxylic acid-(3,3,5,5-tetramethyl)cyclohexyl ester
1-[1-(4′-Cyanobenzyl) imidazol-5-ylmethyl]piperazine-4-carboxylic acid-(3,3-dimethyl)cyclohexyl ester
1-[1-(4′-Cyanobenzyl) imidazol-5-ylmethyl]piperazin-3-one-{4-[2-(1,3,3-trimethylcyclohexane)-1-ethyl]}
1-(1-(4-cyanobenzyl)imidazol-5-ylmethyl)-4-(3,3-dimethylcyclohexyloxycarbonyl)-(cis)-2,6-dimethylpiperazine
1-(4-cyanobenzyl)-5-[1-(3,3-dimethylcylohexylacetyl)piperazin-4-ylmethyl]imidazole
1-[1-(4′-Cyanobenzyl) imidazol-5-ylmethyl]piperazine-4-(2-hydroxy-2-cyclohexylacetamide
1-[1-(4′-Cyanobenzyl) imidazol-5-ylmethyl]piperazine-4-carboxylic acid-[(1R,2S,5R)-2-isopropyl-5-methyl]cyclohexyl ester
1-[1-(4′-Cyanobenzyl) imidazol-5-ylmethyl]piperazine-4-carboxylic acid-(2,6-dimethyl)cyclohexylmethyl ester
1-[1-(4′-Cyanobenzyl) imidazol-5-ylmethyl]piperazine-4-carboxylic acid-(2-tert-butyl)cyclohexyl ester
1-[1-(4′-Cyanobenzyl) imidazol-5-ylmethyl]piperazine-4-carboxylic acid-(3-methyl)cyclohexyl ester
1-[1-(4′-Cyanobenzyl) imidazol-5-ylmethyl] 4-(2,2-dicyclohexyl)acetyl piperazine
1-[1-(4′-Cyanobenzyl) imidazol-5-ylmethyl]piperazine-4-(2,2-dimethyl-3-isobutenylcyclopropane)carboxamide
1-[1-(4-cyanophenyl)methylimidazol-5-ylmethyl]piperazine-4-cyclohexyloxycarbonyl
R/S 1-(4-Cyanobenzyl)-5-[1-(2-hydroxy-2-(adamant-1-yl)ethyl)-2-oxo-piperazin-4-yl-methyl]imidazole
1-(4-Cyanobenzyl)-5-[1-(2-acetyloxy-2-(adamant-1-yl)ethyl)-2-oxo-piperazin-4-yl-methyl]imidazole
1-[1-(4′-Cyanobenzyl) imidazol-5-ylmethyl] piperazine-4-(N-1-adamantyl)carboxamide
1-[1-(4′-Cyanobenzyl) imidazol-5-ylmethyl]-4-(N-1-adamantyl)carbonyl piperazine
1-(4-Cyanobenzyl)-5-[1-(2-(adamant-1-yl)ethyl)-2-oxo-piperazin-4-yl-methyl]imidazole
1-[1-(4′-Cyanobenzyl) imidazol-5-ylmethyl] piperazine-4-carboxylic acid (2-norbomane)methyl ester
1-[1-(4′-Cyanobenzyl) imidazol-5-ylmethyl]-piperazine4-carboxylic acid (2-norbornane)methyl ester
1-[1-(4′-Cyanobenzyl) imidazol-5-ylmethyl]-4-(2-bicyclo-[2.2.2]-octylcarbonyl)piperazine
1-[1-(4′-Cyanobenzyl) imidazol-5-ylmethyl]-4-cis/trans-(2,6,6-trimethylbicyclo[3.1.1 ]heptanecarbonyl)-piperazine
1-[1-(4′-Cyanobenzyl) imidazol-5-ylmethyl]-4-[1-phenyl-1-cyclopentylcarbonyl] piperazine
1-[1-(4′-Cyanobenzyl) imidazol-5-ylmethyl]-4-[cyclohexylphenylacetyl] piperazine
1-[1-(4′-Cyanobenzyl) imidazol-5-ylmethyl]-4-[1-(3-methoxyphenyl)-1-cyclopentylcarbonyl] piperazine
1-[1-(4′-Cyanobenzyl) imidazol-5-ylmethyl]-4-[1-(3-phenoxyphenyl)-1-cyclopentylcarbonyl] piperazine
1-[1-(4′-Cyano-3-fluorobenzyl) imidazol-5-ylmethyl]-4-[1-(3-hydroxyphenyl)-1-cyclohexylcarbonyl] piperazine
1-[1-(4′-Cyanobenzyl) imidazol-5-ylmethyl] piperazine-4-carboxylic acid-(2,6-dimethoxy)benzyl ester
1-[1-(4′-Cyanobenzyl) imidazol-5-ylmethyl] piperazine-4-(DL-2-hydroxy-2-(o-methoxyphenyl)) acetarnide
1-[1-(4′-methylbenzyl) imidazol-5-ylmethyl]-4-[1-(2,6-dimethylbenzyloxycarbonyl] piperazine
1-[1-(4′-Cyanobenzyl) imidazol-5-ylmethyl] piperazine-4-carboxylic acid-(4-nitro)phenyl ester
1-[1-(4′-Cyanobenzyl) imidazol-5-ylmethyl] piperazine-4-(N-3-isopropenyl-1,1-dimethylbenzyl)carboxamide
1-[1-(4′-Cyanobenzyl) imidazol-5-ylmethyl] piperazine-4-(N-2-chlorobenzyl)carboxamide
1-[(4-cyanophenyl)methylimidazol-5-ylmethyl] piperazine-4-2,4-dimethylbenzyloxycarbonyl
1-[1-(4-cyanophenyl)methylimidazol-5-ylmethyl] piperazine-4-(2-methylbenzyloxycarbonyl)
1-[1-(4-cyanophenyl)methylimidazol-5-ylmethyl] piperazine-4-[(3′-methylbenzyloxycarbonyl)
1-[1-(4-cyanophenyl)methylimidazol-5-ylmethyl] piperazine-4-(2′-methoxybenzyloxycarbonyl)
1-[1-(4-cyanophenyl)methylimidazol-5-ylmethyl] piperazine-4-(4′-pyridinemethyloxycarbonyl)
1-[1-(4-cyanophenyl)methylimidazol-5-ylmethyl] piperazine-4-(2′,5′-dimethylbenzyloxycarbonyl)
1-[1-(4′-Cyanobenzyl) imidazol-5-ylmethyl]piperazine-4-carboxylic acid-(2,2-dimethyl)propyl ester
1-(1-(4-cyanobenzyl)imidazol-5-ylmethyl)-4-(N-(1,1,3,3-tetramethyl)-butyl) carboxamide]piperazine
1-[1-(4′-Cyanobenzyl) imidazol-5-ylmethyl]-piperazine-4-carboxylic acid (2,2,5,5-tetramethyl)hexyl ester
1-[1-(4′-Cyanobenzyl) imidazol-5-ylmethyl]-piperazine-4-carboxylic acid (2,2-dimethyl)pent-3-yl ester
1-[1-(4′-Cyanobenzyl) imidazol-5-ylmethyl]piperazine-4-carboxylic acid-(3,3-dimethyl)butyric ester
1-[1-(4′-Cyanobenzyl) imidazol-5-ylmethyl]piperazine-4-(2-hydroxy-4,4-dimethyl)valeramide
1-[1-(4′-Cyanobenzyl) imidazol-5-ylmethyl]piperazine-4-carboxylic acid-(2,2-dimethyl)propyl ester
1-[1-(4′-Cyanobenzyl) imidazol-5-ylmethyl]-4-(2-ethylbutanecarbonyl) piperazine
1-[1-(4′-Cyanobenzyl) imidazol-5-ylmethyl]-piperazine-4-carboxylic acid-(2-t-butoxy)ethyl ester
1-(1-(4-cyanobenzyl)imidazol-5-ylmethyl)-4-(N-(1,1,3,3-tetramethyl)-butyl) carboxamide]piperazine
1-[1-(4′-Cyanobenzyl) imidazol-5-ylmethyl]-piperazine-4-carboxylic acid (2,2,5,5-tetramethyl)hexyl ester
1-[1-(4′-Cyanobenzyl) imidazol-5-ylmethyl]-piperazine-4-carboxylic acid (2,2-dimethyl)pent-3-yl ester
4-[1-(4-Cyanobenzyl)imidazol-5-ylmethyl]-1-(2-methoxyquinolin-4-oyl)piperazine
4-[1-(4-Cyanobenzyl)imidazol-5-ylmethyl]-1-(2-diethylamino-3-ethoxypyrid-5-oyl)piperazine
4-[1-(4-Cyanobenzyl)imidazol-5-ylmethyl]-1-(3-ethylamino-4-isoquinolinoyl)piperazine
4-[1-(4-Cyanobenzyl)imidazol-5-ylmethyl]-1-(5-bromo-1-naphthoyl)piperazine
4-[1-(4-Cyanobenzyl)imidazol-5-ylmethyl]-1-[5-(pent-1-ynyl)-1-naphthoyl]piperazine
4-[1-(4-Cyanobenzyl)imidazol-5-ylmethyl]-1-[5-(prop-1-ynyl)-1-naphthoyl]piperazine
4-[1-(4-Cyanobenzyl)imidazol-5-ylmethyl]-1-(5-propyl-1-naphthoyl)piperazine
4-[1-(4-Cyanobenzyl)imidazol-5-ylmethyl]-1-(4-bromo-3-methylbenzoyl)piperazine
4-[1-(4-Cyanobenzyl)imidazol-5-ylmethyl]-1-[3-methyl-4-(prop-1-ynyl)benzoyl]piperazine
4-[1-(4-Cyanobenzyl)imidazol-5-ylmethyl]-1-(3-methyl-4-pentylbenzoyl)piperazine
4-[1-(4-Cyanobenzyl)imidazol-5-ylmethyl]-1-(2-cyclopropyleth-ynyl-5-methoxybenzoyl)piperazine
4-[1-(4-Cyanobenzyl)imidazol-5-ylmethyl]-1-(5-methoxy-2-pent-1-ynylbenzoyl)piperazine
4-[1-(4-Cyanobenzyl)imidazol-5-ylmethyl]-1-(5-chloro-2-cyclohexylethynylbenzoyl)piperazine
4-[1-(4-Cyanobenzyl)imidazol-5-ylmethyl]-1-(5-chloro-2-cyclohexylethylbenzoyl)piperazine
4-[1-(4-Cyanobenzyl)imidazol-5-ylmethyl]-1-(4-indoloyl)piperazine
4-[1-(4-Cyanobenzyl)imidazol-5-ylmethyl]-1-(3,5-dimethylbenzoyl)piperazine
4-[1-(4-Cyanobenzyl)imidazol-5-ylmethyl]-1-(8-quinolinoyl)piperazine
4-[1-(4-Cyanobenzyl)imidazol-5-ylmethyl]-1-(2-ethoxy-1-naphthoyl)piperazine
4-[1-(4-Cyanobenzyl)imidazol-5-ylmethyl]-1-(2-quinolinoyl)piperazine
4-[1-(4-Cyanobenzyl)imidazol-5-ylmethyl]-1-(3-methoxy-4-methylbenzoyl)piperazine
4-[1-(4-Cyanobenzyl)imidazol-5-ylmethyl]-1-(6-diethylamino-pyrid-2-oyl)piperazine
4-[1-(4-Cyanobenzyl)imidazol-5-ylmethyl]-1-(1-isoquinolinoyl)piperazine
or a pharmaceutically acceptable salt or optical isomer thereof.

5. The assay of claim 2 wherein the radiolabeled GGTase-I inhibitor is selected from a radiolabeled derivative of a compound selected from the group of compounds comprising:
(R,S) 1-[1-(4′-Cyanobenzyl) imidazol-5-ylmethyl]piperazine-4-carboxylic acid-(2R,6R-dimethyl)cyclohexylmethyl ester 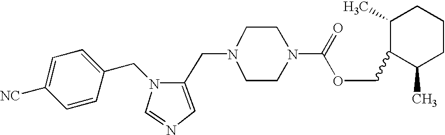
(R,S) 1-[1-(4′-Cyanobenzyl) imidazol-5-ylmethyl]piperazine-4-carboxylic acid-(2R,6S-dimethyl)cyclohexylmethyl ester 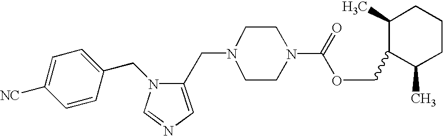
1-[1-(4′-Cyanobenzyl) imidazol-5-ylmethyl]piperazine-4-carboxylic acid-(3,3,5,5-tetramethyl)cyclohexyl ester 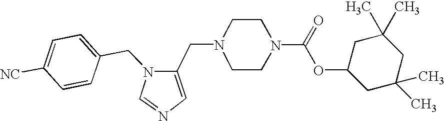
1-(4′-Cyanobenzyl) irnidazol-5-ylmethyl piperazine-4-(2,2-dimethyl-3-isobutenylcyclopropane)carboxamide 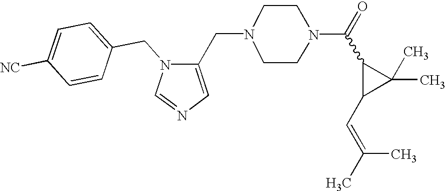
(±)-[1-(4′-Cyanobenzyl) imidazol-5-ylmethyl]piperazine-4-carboxylic acid-(3,3-dimethyl)cyclohexyl ester 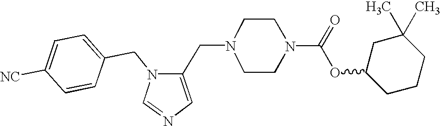
1-[1-(4′-Cyanobenzyl) imidazol-5-ylmethyl] piperazine-4-carboxylic acid-(2,6-dimethoxy)benzyl ester 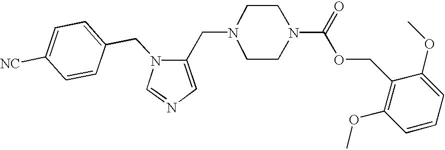
1-[1-(4′-Cyanobenzyl) imidazol-5-ylmethyl]-4-[1-(2,6-dimethylbenzyloxycarbonyl] piperazine 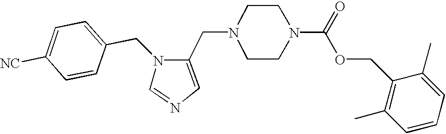
1-[1-(4′-methylbenzyl) imidazol-5-ylmethyl]-4-[1-(2,6-dimethylbenzyloxycarbonyl] piperazine 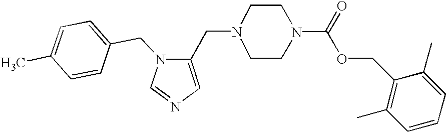
1-[1-(4′-cyanobenzyl) imidazol-5-ylmethyl]-4-[1-(2-ethoxybenzyloxycarbonyl] piperazine 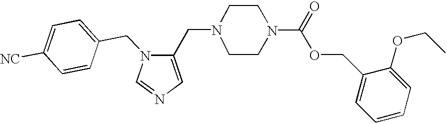
1-[1-(4′-Cyanobenzyl)imidazol-5-ylmethyl]-piperazine-4-(N-2-(ethoxybenzyl)carbamide) 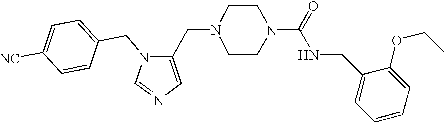
1-[1-1-(4′-Cyanobenzyl) imidazol-5-ylmethyl]-4-{1-[(2-ethoxypyridin-3-yl)methyloxycarbonyl] piperazine 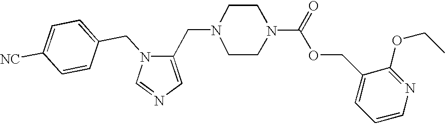
4-[1-(4-Cyanobenzyl)imidazol-5-ylmethyl]-1-[3-methyl-4-(prop-1-ynyl)benzoyl]piperazine 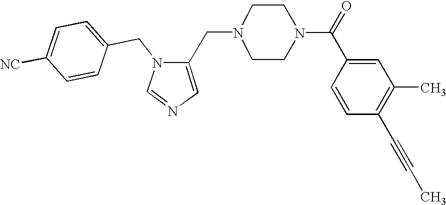
4-[1-(4-Cyanobenzyl)imidazol-5-ylmethyl]-1-(6-diethylamino-pyrid-2-oyl)piperazine 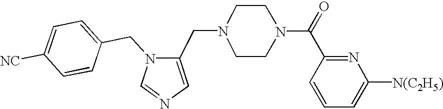
4-[1-(4-Cyanobenzyl)imidazol-5-ylmethyl]-1-(1-naphthoyl)piperazine 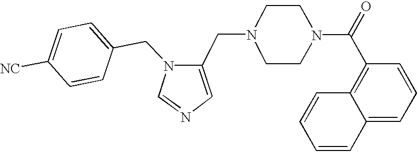
or a pharmaceutically acceptable salt or optical isomer thereof.

6. The assay of claim 2 wherein the radiolabeled GGTase-I inhibitor is: 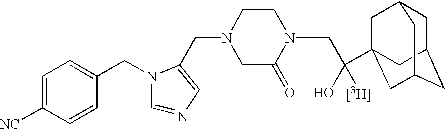
or a pharmaceutically acceptable salt or optical isomer thereof.

7. A compound selected from:
i) a compound of the formula A: 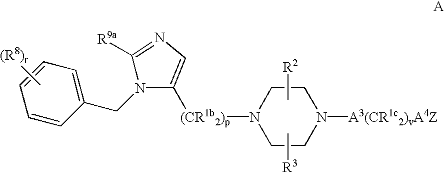
wherein:
R^{1b} is independently selected from:
a) hydrogen,
b) aryl, heterocycle, cycloalkyl, R¹⁰O—, —N(R¹⁰)₂ or C₂-C₆ alkenyl,
c) C₁-C₆ alkyl unsubstituted or substituted by aryl, heterocycle, cycloalkyl, alkenyl, R¹⁰O—, or —N(R¹⁰)₂; _{R}^{1c} is independently selected from:
a) hydrogen,
b) R¹⁰O—, R¹⁰C(O)NR¹⁰—, (R¹⁰)₂NC(O)—, R¹⁰₂N—C(NR¹⁰)—, CN, NO₂, R¹⁰C(O)—, N₃, —N(R¹⁰)₂ or R¹¹OC(O)NR¹⁰—,
c) unsubstituted or substituted C₁-C₆ alkyl wherein the substitutent on the substituted C₁-C₆ alkyl is selected from R¹⁰O—, R¹⁰C(O)NR¹⁰—, (R¹⁰)₂NC(O)—, R¹⁰₂N—C(NR¹⁰)—, CN, R¹⁰C(O)—, N₃, —N(R¹⁰)₂ and R¹¹OC(O)—NR¹⁰—;
R³ is selected from H and CH₃;
R² is selected from 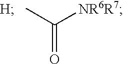
or C₁₋₅ alkyl, unbranched or branched, unsubstituted or substituted with one or more of:
1) aryl,
2) heterocycle,
3) OR⁶,
4) SR^{6a}, SO₂R^{6a}, or
5) 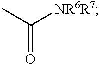
and R² and R³ are optionally attached to the same carbon atom;
R⁶ and R⁷ are independently selected from: H; C₁₋₄ alkyl, C₃₋₆ cycloalkyl, aryl, heterocycle, unsubstituted or substituted with:
a) C₁₋₄ alkoxy,
b) halogen, or
c) aryl or heterocycle;
R^{6a} is selected from: C₁₋₄ alkyl or C₃₋₆ cycloalkyl, unsubstituted or substituted with:
a) C₁₋₄ alkoxy,
b) halogen, or
c) aryl or heterocycle;
R⁸ is independently selected from:
a) hydrogen,
b) C₁-C₆ alkyl, C₂-C₆ alkenyl, C₂-C₆ alkynyl, C₁-C₆ perfluoroalkyl, F, Cl, R¹⁰O—, R¹⁰C(O)NR¹⁰—, CN, NO₂, (R¹⁰)₂N—C(NR¹⁰)—, R¹⁰C(O)—, —N(R¹⁰)₂, or R¹⁰C(O)NR¹⁰—, and
c) C₁-C₆ alkyl substituted by C₁-C₆ perfluoroalkyl, R¹⁰O—, R¹⁰C(O)NR¹⁰—, (R¹⁰)₂N—C(NR¹⁰)—, R¹⁰C(O)—, —N(R¹⁰)₂, or R¹⁰C(O)NR¹⁰—;
R^{9a} is hydrogen or methyl;
R¹⁰ is independently selected from hydrogen, C₁-C₆ alkyl, benzyl and aryl;
R¹¹ is independently selected from C₁-C₆ alkyl and aryl;
A³ is selected from: —C(O)—, —C(O)NR¹⁰— or —C(O)O—;
A⁴ is selected from: bond and —O—;
Z is substituted C₅-C₇ cycloalkyl, wherein the substituted C₅-C₇ cycloalkyl is substituted with one or more C₁₋₄ alkyl moieties and is optionally substituted with one or two moieties selected from the following:
a) C₁₋₄ alkoxy,
b) NR⁶R⁷,
c) C₃₋₆ cycloalkyl,
d) —NR⁶C(O)R⁷,
e) HO,
f) —S(O)ₘR^{6a},
g) halogen, or
h) perfluoroalkyl;
m is 0, 1 or 2;
p is 1,2 or 3;
r is 0 to 5, and
v is 0, 1, 2 or 3;
and wherein at least one radionuclide or ³H-methyl is present in the molecule;
ii) a compound of the formula B: 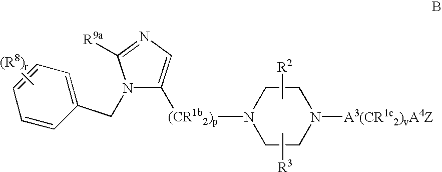
wherein:
R^{1b}, R^{1c}, R², R³, R⁶, R⁷, R^{6a}, R⁸, R^{9a}, R¹⁰ , R¹¹, A³, A⁴, m, p, r and v are as defined above for the compound of the formula A;
Z is an unsubstituted or substituted C₇-C₁₀ multicyclic alkyl ring, wherein the substituted C₇-C₁₀ multicyclic alkyl ring is substituted with one or two moieties selected from the following:
a) C₁₋₄ alkoxy,
b) NR⁶R⁷,
c) C₃₋₆ cycloalkyl,
d) —NR⁶C(O)R⁷,
e) HO,
f) —S(O)ₘR^{6a},
g) halogen,
h) perfluoroalkyl, and
i) C₁₋₄alkyl;
C₇-C₁₀ multicyclic alkyl ring is selected from: 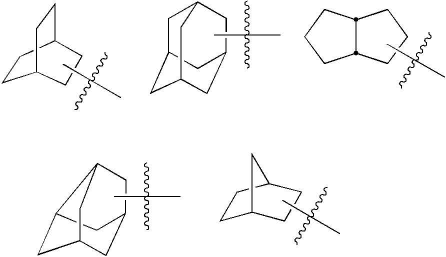
and wherein at least one radionuclide or ³H-methyl is present in the molecule;
iii) a compound of the formula C: 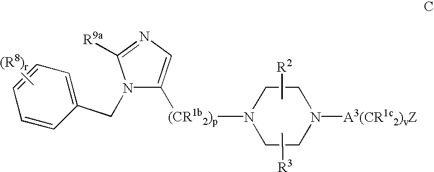
wherein:
R^{1c}, R³, R⁶, R⁷, R^{6a}, R⁸, R¹⁰, R¹¹, m, p and r are as defined above for the compound of the formula A;
R^{1b} is independently selected from:
a) hydrogen,
b) aryl, heterocycle, cycloalkyl, CN, R¹⁰O—, R¹⁰NC(O)—, —N(R¹⁰)₂ or C₂-C₆ alkenyl,
c) C₁-C₆ alkyl unsubstituted or substituted by aryl, heterocycle, cycloalkyl, alkenyl, R¹⁰O—, or —N(R¹⁰)₂;
R² is selected from 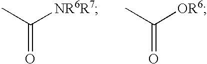
or C₁₋₅ alkyl, unbranched or branched, unsubstituted or substituted with one ormoreof:
1) aryl,
2) heterocycle,
3) OR⁶,
4) SR^{6a}, SO₂R^{6a}, or
5) 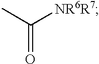
and R² and R³ are optionally attached to the same carbon atom;
R^{9a} is hydrogen, C₁-C₆ alkyl or chloro;
A³ is selected from: —C(O)—, —C(O)NR¹⁰—, —C(O)O— and S(O)ₘ;
Z is unsubstituted or substituted phenyl, unsubstituted or substituted napthyl, unsubstituted or substituted pyridyl, unsubstituted or substituted quinoline or unsubstituted or substituted 1,2 methylenedioxybenzene; and
v is 0, 1, 2 or 3; provided that v is not 0 if A³ is —C(O)— or S(O)ₘ;
and wherein at least one radionuclide or ³H-methyl is present in the molecule;
iv) a compound of the formula D: 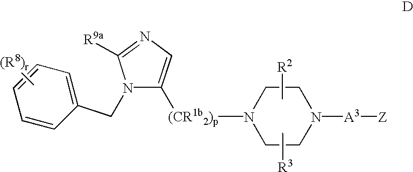
wherein:
R^{1b}, R², R³, R⁶, R⁷, R^{6a}, R⁸ R^{9a}, R¹⁰, R¹¹, A³, m, p, r and v are as defined above for the compound of the formula A;
Z is unsubstituted or substituted C₅-C₁₀ alkyl, wherein the substituted C₅-C₁₀ alkyl is substituted with one or two moieties selected from the following:
a) C₁₋₄ alkoxy,
b) NR⁶R⁷,
c) C₃₋₆ cycloalkyl,
d) —NR⁶C(O)R⁷,
e) —OR¹⁰,
f) —S(O)ₘR^{6a},
g) halogen, or
h) perfluoroalkyl;
and wherein at least one radionuclide or ³H-methyl is present in the molecule;
v) a compound of the formula E: 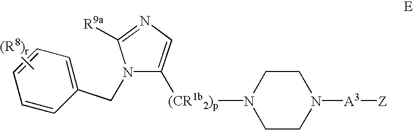
wherein:
R^{1b}, R⁸, R¹¹, p and r are as defined above for the compound of the formula A;
R^{9a} is hydrogen, C₁-C₆ alkyl or chloro;
R¹⁰ is independently selected from hydrogen, C₁-C₆ alkyl, benzyl and aryl;
A³ is —C(O)—;
Z is unsubstituted or substituted phenyl, unsubstituted or substituted napthyl, unsubstituted or substituted pyridyl, unsubstituted or substituted 2,3-dihydrobenzofuran, unsubstituted or substituted quinoline or unsubstituted or substituted isoquinoline;
and wherein at least one radionuclide or ³H-methyl is present in the molecule;
or a pharmaceutically acceptable salt thereof.

8. A radiolabeled derivative of a compound selected from the group of compounds comprising:
1-[1-(4′-Cyanobenzyl) imidazol-5-ylmethyl]piperazine-4-carboxylic acid-(3,3,5,5-tetramethyl)cyclohexyl ester
1-[1-(4′-Cyanobenzyl) imidazol-5-ylmethyl]piperazine-4-carboxylic acid-(3,3-dimethyl)cyclohexyl ester
1-[1-(4′-Cyanobenzyl) imidazol-5-ylmethyl]piperazin-3-one-{4-[2-(1,3,3-trimethylcyclohexane)-1-ethyl]}
1-(1-(4-cyanobenzyl)imidazol-5-ylmethyl)-4-(3,3-dimethylcyclohexyloxycarbonyl)-(cis)-2,6-dimethylpiperazine
1-(4-cyanobenzyl)-5-[1-(3,3-dimethylcylohexylacetyl)piperazin-4-ylmethyl]imidazole 1-[1-(4′-Cyanobenzyl) imidazol-5-ylmethyl]piperazine-4-(2-hydroxy-2-cyclohexylacetamide
1-[1-(4′-Cyanobenzyl) imidazol-5-ylmethyl]piperazine-4-carboxylic acid-[(1R,2S,5R)-2-isopropyl-5-methyl]cyclohexyl ester
1-[1-(4′-Cyanobenzyl) imidazol-5-ylmethyl]piperazine-4-carboxylic acid-(2,6-dimethyl)cyclohexylmethyl ester
1-[1-(4′-Cyanobenzyl) imidazol-5-ylmethyl]piperazine-4-carboxylic acid-(2-tertbutyl)cyclohexyl ester
1-[1-(4′-Cyanobenzyl) imidazol-5-ylmethyl]piperazine-4-carboxylic acid-(3-methyl )cyclohexyl ester
1-[1-(4′-Cyanobenzyl) imidazol-5-ylmethyl] 4-(2,2-dicyclohexyl)acetyl piperazine
1-[1-(4′-Cyanobenzyl) imidazol-5-ylmethyl]piperazine-4-(2,2-dimethyl-3-isobutenylcyclopropane)carboxamide
1-[1-(4-cyanophenyl)methylimidazol-5-ylmethylpiperazine-4-cyclohexyloxycarbonyl
R/S 1-(4-Cyanobenzyl)-5-[1-(2-hydroxy-2-(adamant-1-yl)ethyl)-2-oxo-piperazin-4-yl-methyl]imidazole
1-(4-Cyanobenzyl)-5-[1-(2-acetyloxy-2-(adamant-1-yl)ethyl)-2-oxo-piperazin-4-yl-methyl]imidazole
1-[1-(4′-Cyanobenzyl) imidazol-5-ylmethyl] piperazine-4-(N-1-adamantyl)carboxamide
1-[1-(4′-Cyanobenzyl) imidazol-5-ylmethyl]-4-(N-1-adamantyl)carbonyl piperazine
1-(4-Cyanobenzyl)-5-[1-(2-(adamant-1-yl)ethyl)-2-oxo-piperazin-4-yl-methyl]imidazole
1-[1-(4′-Cyanobenzyl) imidazol-5-ylmethyl] piperazine-4-carboxylic acid (2-norbomane)methyl ester
1-[1-(4′-Cyanobenzyl) imidazol-5-ylmethyl]-piperazine-4-carboxylic acid (2-norbomane)methyl ester
1-[1-(4′-Cyanobenzyl) imidazol-5-ylmethyl]-4-(2-bicyclo-[2.2.2]-octylcarbonyl)piperazine
1-[1-(4′-Cyanobenzyl) imidazol-5-ylmethyl]-4-cis/trans-(2,6,6-trimethylbicyclo[3.1.1]heptanecarbonyl)-piperazine
1-[1-(4′-Cyanobenzyl) imidazol-5-ylmethyl]-4-[1-phenyl-1-cyclopentylcarbonyl] piperazine
1-[1-(4′-Cyanobenzyl) imidazol-5-ylmethyl]-4-[cyclohexylphenylacetyl] piperazine
1-[1-(4′-Cyanobenzyl) imidazol-5-ylmethyl]-4-[1-(3-methoxyphenyl)-1-cyclopentylcarbonyl] piperazine
1-[1-(4′-Cyanobenzyl) imidazol-5-ylmethyl]-4-[1-(3-phenoxyphenyl)-1-cyclopentylcarbonyl] piperazine
1-[1-(4′-Cyano-3-fluorobenzyl) imidazol-5-ylmethyl]-4-[1-(3-hydroxyphenyl)-1-cyclohexylcarbonyl] piperazine
1-[1-(4′-Cyanobenzyl) imidazol-5-ylmethyl] piperazine-4-carboxylic acid-(2,6-dimethoxy)benzyl ester
1-[1-(4′-Cyanobenzyl) imidazol-5-ylmethyl] piperazine-4-(DL-2-hydroxy-2-(o-methoxyphenyl)) acetamide
1-[1-(4′-methylbenzyl) imidazol-5-ylmethyl]-4-[1-(2,6-dimethylbenzyloxycarbonyl] piperazine
1-[1-(4′-Cyanobenzyl) imidazol-5-ylmethyl] piperazine-4-carboxylic acid-(4-nitro)phenyl ester
1-[1-(4′-Cyanobenzyl) imidazol-5-ylmethyl] piperazine-4-(N-3-isopropenyl-1,1-dimethylbenzyl)carboxamide
1-[1-(4′-Cyanobenzyl) imidazol-5-ylmethyl] piperazine-4-(N-2-chlorobenzyl)carboxamide
1-[(4-cyanophenyl)methylimidazol-5-ylmethyl] piperazine-4-2,4-dimethylbenzyloxycarbonyl
1-[1-(4-cyanophenyl)methylimidazol-5-ylmethyl] piperazine-4-(2-methylbenzyloxycarbonyl)
1-[1-(4-cyanophenyl)methylimidazol-5-ylmethyl] piperazine-4-[(3′-methylbenzyloxycarbonyl)
1-[1-(4-cyanophenyl)methylimidazol-5-ylmethyl] piperazine-4-(2′-methoxybenzyloxycarbonyl)
1-[1-(4-cyanophenyl)methylimidazol-5-ylmethyl] piperazine-4-(4′-pyridinemethyloxycarbonyl)
1-[1-(4-cyanophenyl)methylimidazol-5-ylmethyl] piperazine-4-(2′,5′-dimethylbenzyloxycarbonyl)
1-[1-(4′-Cyanobenzyl) imidazol-5-ylmethyl]piperazine-4-carboxylic acid-(2,2-dimethyl)propyl ester
1-(1-(4-cyanobenzyl)imidazol-5-ylmethyl)-4-(N-(1,1,3,3-tetramethyl)-butyl) carboxamide]piperazine
1-[1-(4′-Cyanobenzyl) imidazol-5-ylmethyl]-piperazine-4-carboxylic acid (2,2,5,5-tetramethyl)hexyl ester
1-[1-(4′-Cyanobenzyl) imidazol-5-ylmethyl]-piperazine-4-carboxylic acid (2,2
-dimethyl)pent-3-yl ester
1-[1-(4′-Cyanobenzyl) imidazol-5-ylmethyl]piperazine-4-carboxylic acid-(3,3-dimethyl)butyric ester
1-[1-(4′-Cyanobenzyl) imidazol-5-ylmethyl]piperazine-4-(2-hydroxy-4,4-dimethyl)valeramide
1-[1-(4′-Cyanobenzyl) imidazol-5-ylmethyl]piperazine-4-carboxylic acid-(2,2-dimethyl)propyl ester
1-[1-(4′-Cyanobenzyl) imidazol-5-ylmethyl]-4-(2-ethylbutanecarbonyl) piperazine
1-[1-(4′-Cyanobenzyl) imidazol-5-ylmethyl]-piperazine-4-carboxylic acid-(2-t-butoxy)ethyl ester
1-(1-(4-cyanobenzyl)imidazol-5-ylmethyl)-4-(N-(1,1,3 ,3-tetramethyl)-butyl) carboxamide]piperazine
1-[1-(4′-Cyanobenzyl) imidazol-5-ylmethyl]-piperazine-4-carboxylic acid (2,2,5,5-tetramethyl)hexyl ester
1-[1-(4′-Cyanobenzyl) imidazol-5-ylmethyl]-piperazine-4-carboxylic acid (2,2-dimethyl)pent-3-yl ester
4-[1-(4-Cyanobenzyl)imidazol-5-ylmethyl] 1-(2-methoxyquinolin-4-oyl)piperazine
4-[1-(4-Cyanobenzyl)imidazol-5-ylmethyl]-1-(2-diethylamino-3-ethoxypyrid-5-oyl)piperazine
4-[1-(4-Cyanobenzyl)imidazol-5-ylmethyl]-1-(3-ethylamino-4-isoquinolinoyl)piperazine
4-[1-(4-Cyanobenzyl)imidazol-5-ylmethyl]-1-(5-bromo-1-naphthoyl)piperazine
4-[1-(4-Cyanobenzyl)imidazol-5-ylmethyl]-1-[5-(pent-1-ynyl)-1-naphthoyl]piperazine
4-[1-(4-Cyanobenzyl)imidazol-5-ylmethyl]-1-[5-(prop-1-ynyl)-1-naphthoyl]piperazine
4-[1-(4-Cyanobenzyl)imidazol-5-ylmethyl]-1-(5-propyl-1-naphthoyl)piperazine
4-[1-(4-Cyanobenzyl)imidazol-5-ylmethyl]-1-(4-bromo-3-methylbenzoyl)piperazine
4-[1-(4-Cyanobenzyl)imidazol-5-ylmethyl]-1-[3-methyl-4-(prop-1-ynyl)benzoyl]piperazine
4-[1-(4-Cyanobenzyl)imidazol-5-ylmethyl]-1-(3-methyl-4-pentylbenzoyl)piperazine
4-[1-(4-Cyanobenzyl)imidazol-5-ylmethyl]-1-(2-cyclopropyleth-ynyl-5-methoxybenzoyl)piperazine
4-[1-(4-Cyanobenzyl)imidazol-5-ylmethyl]-1-(5-methoxy-2-pent-1-ynylbenzoyl)piperazine
4-[1-(4-Cyanobenzyl)imidazol-5-ylmethyl]-1-(5-chloro-2-cyclohexylethynylbenzoyl)piperazine
4-[1-(4-Cyanobenzyl)imidazol-5-ylmethyl]-1-(5-chloro-2-cyclohexylethylbenzoyl)piperazine
4-[1-(4-Cyanobenzyl)imidazol-5-ylmethyl]-1-(4-indoloyl)piperazine
4-[1-(4-Cyanobenzyl)imidazol-5-ylmethyl]-1-(3 ,5-dimethylbenzoyl)piperazine
4-[1-(4-Cyanobenzyl)imidazol-5-ylmethyl]-1-(8-quinolinoyl)piperazine
4-[1-(4-Cyanobenzyl)imidazol-5-ylmethyl]-1-(2-ethoxy-1-naphthoyl)piperazine
4-[1-(4-Cyanobenzyl)imidazol-5-ylmethyl]-1-(2-quinolinoyl)piperazine 4-[1-(4-Cyanobenzyl)imidazol-5-ylmethyl]-1-(3-methoxy-4-methylbenzoyl)piperazine
4-[1-(4-Cyanobenzyl)imidazol-5-ylmethyl]-1-(6-diethylamino-pyrid-2-oyl)piperazine
4-[1-(4-Cyanobenzyl)imidazol-5-ylmethyl]-1-(1-isoquinolinoyl)piperazine
or a pharmaceutically acceptable salt thereof.

9. A radiolabeled derivative of a compound selected from the group of compounds comprising:
(R,S) 1-[1-(4′-Cyanobenzyl) imidazol-5-ylmethyl]piperazine-4-carboxylic acid-(2 R,6R-dimethyl)cyclohexylmethyl ester 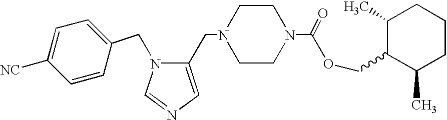
(R,S) 1-[1-(4′-Cyanobenzyl) imidazol-5-ylmethyl]piperazine-4-carboxylic acid-(2R,6S-dimethyl)cyclohexylmethyl ester 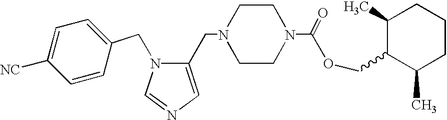
1-[1-(4′-Cyanobenzyl) imidazol-5-ylmethyl]piperazine-4-carboxylic acid-(3,3,5,5-tetramethyl)cyclohexyl ester 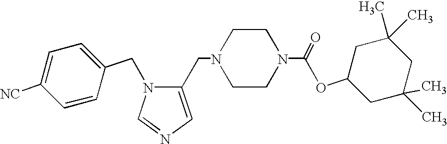
1-(4-Cyanobenzyl)imidazol-5-ylmethyl piperazine-4-(2,2-dimethyl-3-isobutenylcyclopropane)carboxamide 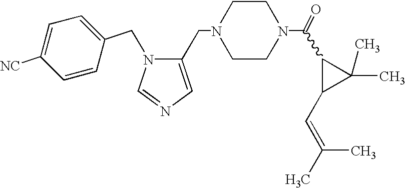
(±)(1-[1-(4′-Cyanobenzyl) irnidazol-5-ylmethyl]piperazine-4-carboxylic acid-(3,3-dimethyl)cyclohexyl ester 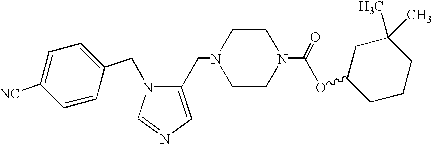
1-[1-(4′-Cyanobenzyl) imidazol-5-ylmethyl] piperazine-4-carboxylic acid-(2,6-dimethoxy)benzyl ester 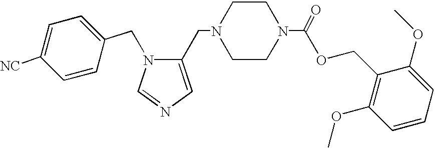
1-[1-(4′-Cyanobenzyl) imidazol-5-ylmethyl]-4-[1-(2,6-dimethylbenzyloxycarbonyl] piperazine 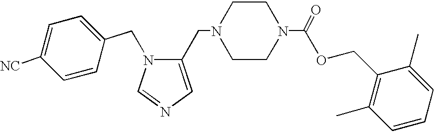
1-[1-(4′-methylbenzyl) imidazol-5-ylmethyl]-4-[1-(2,6-dimethylbenzyloxycarbonyl] piperazine 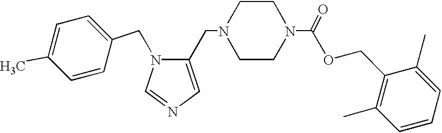
1-[1-(4′-cyanobenzyl) imidazol-5-ylmethyl]-4-[1-(2-ethoxybenzyloxycarbonyl] piperazine 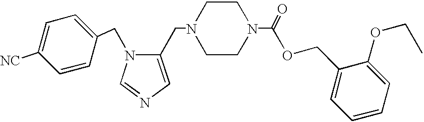
1-[1-(4′-Cyanobenzyl)imidazol-5-ylmethyl]-piperazine-4-(N-2-(ethoxybenzyl)carbamide) 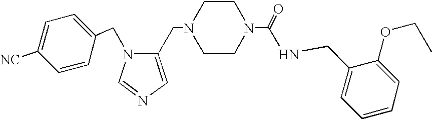
1-[-1-1-(4′-Cyanobenzyl) irnidazol-5-ylmethyl]-4-{1-[(2-ethoxypyridin-3-yl)methyloxycarbonyl] piperazine 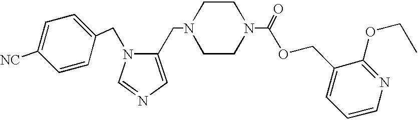
4-[1-(4-Cyanobenzyl)imidazol-5-ylmethyl]-1-[3-methyl-4-(prop-1-ynyl)benzoyl]piperazine [chemical structure]
4-[1-(4-Cyanobenzyl)imidazol-5-ylmethyl]-1-(6-diethylamino-pyrid-2-oyl)piperazine [chemical structure]
4-[1-(4-Cyanobenzyl)imidazol-5-ylmethyl]-1-(1-naphthoyl)piperazine [chemical structure]
or a pharmaceutically acceptable salt or optical isomer thereof.

10. A compound which is: 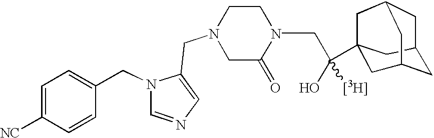
or a pharmaceutically acceptable salt or optical isomer thereof.
